(19) United States
(12) Patent Application Publication
(10) Pub. No.: US 2024/0350034 A1
(43) Pub. Date: Oct. 24, 2024

(54) ESTIMATION DEVICE, INFORMATION PRESENTATION SYSTEM, ESTIMATION METHOD, AND RECORDING MEDIUM

(71) Applicant: NEC Corporation, Minato-ku, Tokyo (JP)

(72) Inventors: Fumiyuki NIHEY, Tokyo (JP); Kentaro Nakahara, Tokyo (JP)

(73) Assignee: NEC Corporation, Minato-ku, Tokyo (JP)

(21) Appl. No.: 18/579,911

(22) PCT Filed: Sep. 27, 2021
(86) PCT No.: PCT/JP2021/035229

§ 371 Date: Jan. 17, 2024

(51) Int. Cl. A61B 5/11; A61B 5/00

(52) U.S. Cl. CPC A61B 5/112; A61B 5/7275

(57) ABSTRACT

An estimation device that includes an acquisition unit that acquires sensor data measured in accordance with walking of the user, and physical data of the user, a storage unit that stores an estimation model and the physical data, the estimation model outputting care-related information in response to inputs of a feature quantity extracted from the sensor data and the physical data, an estimation unit that inputs the feature quantity extracted from the sensor data of the user and the physical data into the estimation model to estimate the care-related information of the user, and an output unit that outputs the estimated care-related information of the user.

### TECHNICAL FIELD

The present disclosure relates to an estimation device or the like that estimates care-related information based on a gait.

### BACKGROUND ART

As if in response to the growing interest in healthcare, a service that provides information corresponding to a feature (also referred to as gait) included in a walking pattern to a user has attracted attention. For example, a technique for analyzing a gait of the user using sensor data measured by a sensor mounted on footwear such as shoes has been developed. By estimating the physical condition based on the information regarding the gait, an appropriate measure can be taken according to the sign appearing in walking. For example, by estimating the progress degree of frailty indicating decrepitude of the body and mind due to aging, the risk of falling down, and the like based on the information regarding gait, it may be possible for the elderly to avoid from becoming in need of care due to unexpected falling or the like. NPLs 1 to 5 disclose several examples of the relationship between the risk of frailty or falling and the gait.

NPL 1 discloses initial symptoms and phenotypes of frailty. NPL 1 discloses a diagram relating to a frail cycle (FIG. 1 of Non-Patent Document). NPL 1 indicates that frailty progresses as factors such as weight, amount of activity, walking speed, muscle strength, and balance are mutually related. According to NPL 1, a decrease in walking speed and a balance disorder are likely to occur with a decrease in muscle strength.

NPL 2 discloses a difference in characteristics appearing in the walking speed depending on the presence or absence of the symptom of frailty (Table 4 of NPL 2). According to NPL 2, regarding a subject who has a symptom of frailty, the walking speed tends to decrease, and the distribution of the walking speed tends to increase.

NPL 3 discloses the prevalence of frailty according to age group (Table 1 of NPL 3). According to NPL 3, the prevalence of frailty is 10% or less in the age group from 65 to 74 years old, and reaches 35% in the age group of 80 years old or more.

NPL 4 discloses features appearing in the gait of a person who has fallen (easily falling person) and a person who has not fallen (non-falling person) in a specific verification period (FIG. 1 of NPL 4). According to NPL 4, the stride time of a person who easily falls is more varied than that of a person who does not fall.

NPL 5 discloses the falling rate according to age group and gender (FIG. 3 of NPL 5). According to NPL 5, the falling rate of men is slightly higher than 10% in the age group from 65 to 69 years old, whereas it is higher than 30% in the age group of 85 years old or more. The falling rate of men is slightly more than 10% in the age group from 65 to 69 years old, and reaches 50% in the age group of 85 years old or more.

PTL 1 discloses an information processing device that extracts a feature quantity used for individual identification using motion information of a foot of a user. The device of PTL 1 extracts a feature quantity using motion information of a foot measured by a motion measurement device provided on a foot of a user.

PTL 2 discloses an exercise ability evaluation system that evaluates an exercise ability of a subject during physical movements in daily life for care and care prevention. The system of PTL 2 measures the position and angle of the body part during the body motion of the subject with a motion capture device or the like. The system of PTL 2 calculates the exercise capacity of the body part of the subject in time series based on the measurement result. The system of PTL 2 calculates a specific value of the exercise capacity of the body part based on the exercise capacity of the body part of the subject calculated in time series. The system of PTL 2 evaluates the calculated specific value of the exercise capacity of the body part as the exercise capacity of the subject during the body motion in daily life.

PTL 3 discloses a user support system that generates user support data based on measurement data measured by a measurement device such as a wearable device worn on a wrist or an arm of a user. The system of PTL 3 generates user assistance data for indicating a state of a frailty level of a user.

### CITATION LIST

### Patent Literature

PTL 1: WO 2020/240751 A1
PTL 2: JP 2019-154489 A
PTL 3: JP 6815055 B2

### Non Patent Literature

NPL 1: Q. Xue. et al., “Initial Manifestations of Frailty Criteria and the Development of Frailty Phenotype in the Women's Health and Aging Study II”, Journal of Gerontology: MEDICAL SCIENCES, 2008, 63A(9), pp. 984-990.
NPL 2: M. Schwenk, et al., “Wearable Sensor-Based In-Home Assessment of Gait, Balance, and Physical Activity for Discrimination of Frailty Status: Baseline Results of the Arizona Frailty Cohort Study”, Gerontology, 2015, 61(3), pp. 258-67.
NPL 3: H. Shimada, et al., “Combined Prevalence of Frailty and Mild Cognitive Impairment in a Population of Elderly Japanese People”, Journal of the American Medical Directors Association, 2013, pp. 1-7.
NPL 4: J. Hausdorff, et al., “Gait Variability and Fall Risk in Community-Living Older Adults: A 1-Year Prospective Study”, Arch Phys Med Rehabil, Vol 82, August 2001, pp. 1050-1056.
NPL 5: C. Pearson, et al., “Understanding seniors' risk of falling and their perception of risk”, Statistics Canada, Catalogue no. 82-624-X, Health at a Glance, October 2014, pp. 1-11.

### SUMMARY OF INVENTION

### Technical Problem

According to the method of PTL 1, a feature quantity used for individual identification can be extracted by using the exercise information of the user's foot. However, the method of PTL 1 cannot estimate a symptom of frailty or the like.

In the method of PTL 2, it is possible to classify an elderly person who needs immediate rehabilitation intervention or an elderly person who may need rehabilitation intervention in the near future based on specific values of the upper body, the left lower body, and the right lower body. However, in the method of PTL 2, it is necessary to measure the position and angle of the body part during the body motion with a motion capture device or the like. Therefore, in the method of PTL 2, it is not possible to estimate the degree of progress of user's frailty and the falling risk in general daily life.

According to the method of PTL 3, it is possible to reduce the probability of occurrence of the risk by providing the user with a notification for frailty prevention or the like according to the user support data. In the method of PTL 3, based on a comparison result obtained by comparing a measurement value with a reference value, user support data indicating a frail level or a symptom level associated with the comparison result is generated. However, the technique of PTL 3 does not disclose a specific procedure for estimating the frailty level or the symptom level.

That is, by the methods of PTLs 1 to 3, it is not possible to estimate the care-related information according to the physical condition of the user based on the gait of the user.

An object of the present disclosure is to provide an estimation device or the like capable of estimating care-related information according to a physical condition of a user based on a gait of the user.

### Solution to Problem

An estimation device according to an aspect of the present disclosure includes an acquisition unit that acquires sensor data measured in accordance with walking of the user, and physical data of the user, a storage unit that stores an estimation model and the physical data, the estimation model outputting care-related information in accordance with the input of a feature quantity extracted from the sensor data and the physical data, an estimation unit that inputs the feature quantity extracted from the sensor data of the user and the physical data into the estimation model to estimate the care-related information of the user, and an output unit that outputs the estimated care-related information of the user.

In an estimation method of one aspect of the present disclosure, sensor data measured according to walking of a user and physical data of the user are acquired, a feature quantity extracted from the sensor data of the user and the physical data are input to an estimation model that outputs care-related information according to inputs of the feature quantity extracted from the sensor data and the physical data to estimate the care-related information of the user, and the estimated care-related information of the user is output.

A program according to an aspect of the present disclosure causes a computer to execute acquiring sensor data measured according to walking of a user and physical data of the user, inputting a feature quantity extracted from the sensor data of the user and the physical data to an estimation model that outputs care-related information according to inputs of the feature quantity extracted from the sensor data and the physical data to estimate the care-related information of the user, and outputting the estimated care-related information of the user.

### Advantageous Effects of Invention

According to the present disclosure, it is possible to provide an estimation device or the like capable of estimating care-related information according to a physical condition of a user based on the gait of the user.

### BRIEF DESCRIPTION OF DRAWINGS

FIG. 1 is a block diagram illustrating an example of a configuration of an information presentation system according to a first example embodiment.

FIG. 2 is a conceptual diagram illustrating an arrangement example of a measurement device of the information presentation system according to the first example embodiment.

FIG. 3 is a conceptual diagram for explaining a gait cycle used in the information presentation system according to the first example embodiment.

FIG. 4 is a conceptual diagram for explaining an example of a frail cycle related to the information presentation system according to the first example embodiment.

FIG. 5 is a conceptual diagram for explaining an example of a walking speed distribution related to the information presentation system according to the first example embodiment.

FIG. 6 is a conceptual diagram for explaining an example of a frailty prevalence regarding the information presentation system according to the first example embodiment.

FIG. 7 is a graph for explaining a derivation example of constants of a curve related to a frailty prevalence used by the information presentation system according to the first example embodiment.

FIG. 8 is a graph illustrating an example of a function used for estimating a frailty prevalence by the information presentation system according to the first example embodiment.

FIG. 9 is a block diagram illustrating an example of a configuration of a measurement device of the information presentation system according to the first example embodiment.

FIG. 10 is a block diagram illustrating an example of a configuration of an estimation device of the information presentation system according to the first example embodiment.

FIG. 11 is a conceptual diagram for explaining a stride length calculated by the estimation device included in the information presentation system according to the first example embodiment.

FIG. 12 is a graph for explaining an example of a walking event detected by the estimation device included in the information presentation system according to the first example embodiment.

FIG. 13 is a flowchart for explaining an example of estimation of care-related information by the estimation device included in the information presentation system according to the first example embodiment.

FIG. 14 is a flowchart for explaining an example of gait parameter calculation processing by the estimation device included in the information presentation system according to the first example embodiment.

FIG. 15 is a conceptual diagram for explaining Application Example 1 of the information presentation system according to the first example embodiment.

FIG. 16 is a block diagram illustrating an example of a configuration of an information presentation system according to a second example embodiment.

FIG. 17 is a conceptual diagram for explaining an example of a walking speed distribution related to the information presentation system according to the second example embodiment.

FIG. 18 is a conceptual diagram for explaining an example of stride time variation related to the information presentation system according to the second example embodiment.

FIG. 19 is a conceptual diagram for explaining an example of falling easiness regarding the information presentation system according to the second example embodiment.

FIG. 20 is a graph for explaining a derivation example of a constant of a curve related to a falling rate used by the information presentation system according to the second example embodiment.

FIG. 21 is a graph illustrating an example of a function used for estimation of the falling rate by the information presentation system according to the second example embodiment.

FIG. 22 is a block diagram illustrating an example of a configuration of an estimation device of the information presentation system according to the second example embodiment.

FIG. 23 is a flowchart for explaining an example of estimation of care-related information by the estimation device included in the information presentation system according to the second example embodiment.

FIG. 24 is a flowchart for explaining an example of gait parameter calculation processing by the estimation device included in the information presentation system according to the second example embodiment.

FIG. 25 is a conceptual diagram for explaining Application Example 2 of the information presentation system according to the second example embodiment.

FIG. 26 is a block diagram illustrating an example of a configuration of an estimation device according to a third example embodiment.

FIG. 27 is a block diagram illustrating an example of a hardware configuration that executes control and processing according to each example embodiment.

### EXAMPLE EMBODIMENT

Hereinafter, example embodiments of the present invention will be described with reference to the drawings. However, the example embodiments described below may be technically limited for carrying out the present invention, but the scope of the invention is not limited to the following. In all the drawings used in the following description of the example embodiment, the same reference numerals are given to the same parts unless there is a particular reason. In the following example embodiments, repeated description of similar configurations and operations may be omitted.

### First Example Embodiment

An information presentation system according to a first example embodiment will be described with reference to the drawings. The information presentation system according to the present example embodiment measures features (also referred to as gait) included in the walking pattern of the user. The information presentation system according to the present example embodiment estimates the care-related information of the user by analyzing the measured gait. In the present example embodiment, an example in which care-related information is estimated based on sensor data regarding a motion of a foot will be described. In the present example embodiment, a system in which the right foot is a reference foot and the left foot is an opposite foot will be described. The method of the present example embodiment can also be applied to a system in which the left foot is a reference foot and the right foot is an opposite foot.

### (Configuration)

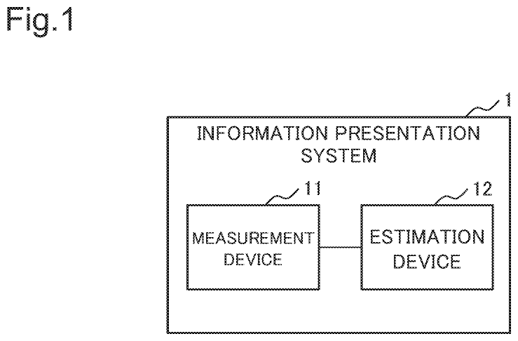

FIG. 1 is a block diagram illustrating a configuration of an information presentation system 1 according to the present example embodiment. The information presentation system 1 includes a measurement device 11 and an estimation device 12. The measurement device 11 and the estimation device 12 may be connected by wire or wirelessly. The measurement device 11 and the estimation device 12 may be configured by a single device. The information presentation system 1 may include only the estimation device 12 except for the measurement device 11 from the configuration of the information presentation system 1.

The measurement device 11 is installed on the foot portion. For example, the measurement device 11 is installed in a footwear such as shoes. In the present example embodiment, an example in which the measurement device 11 is disposed at a position on the back side of the arch of foot will be described. The measurement device 11 includes an acceleration sensor and an angular velocity sensor. The measurement device 11 measures physical quantities such as acceleration measured by the acceleration sensor (also referred to as spatial acceleration) and an angular velocity measured by the angular velocity sensor (also referred to as spatial angular velocity) as physical quantities regarding the movement of the foot of the user wearing the footwear. The physical quantity regarding the movement of the foot measured by the measurement device 11 includes a speed, an angle, and a position (trajectory) calculated by integrating the acceleration and the angular velocity. The measurement device 11 converts the measured physical quantity into digital data (also referred to as sensor data). The measurement device 11 transmits the converted sensor data to the estimation device 12. For example, the measurement device 11 is connected to the estimation device 12 via a mobile terminal (not illustrated) carried by the user.

A mobile terminal (not illustrated) is a communication device that can be carried by a user. For example, the mobile terminal is a portable communication device having a communication function, such as a smartphone, a smart watch, or a mobile phone. The mobile terminal receives, from the measurement device 11, sensor data regarding the movement of the foot of the user. The mobile terminal transmits the received sensor data to a server, a cloud, or the like on which the estimation device 12 is mounted. The functions of the estimation device 12 may be achieved by application software or the like installed in the mobile terminal. In this case, the mobile terminal processes the received sensor data by application software or the like installed therein.

The measurement device 11 is achieved by, for example, an inertial measurement device including an acceleration sensor and an angular velocity sensor. An example of the inertial measurement device is an inertial measurement unit (IMU). The IMU includes an acceleration sensor that measures accelerations in three axial directions and an angular velocity sensor that measures angular velocitys around three axes. The measurement device 11 may be achieved by an inertial measurement device such as a vertical gyro (VG) or an attitude heading (AHRS). The measurement device 11 may be achieved by a Global Positioning System/Inertial Navigation System (GPS/INS). The measurement device 11 is not limited to the inertial measurement device as long as it can measure the physical quantity related to the movement of the foot.

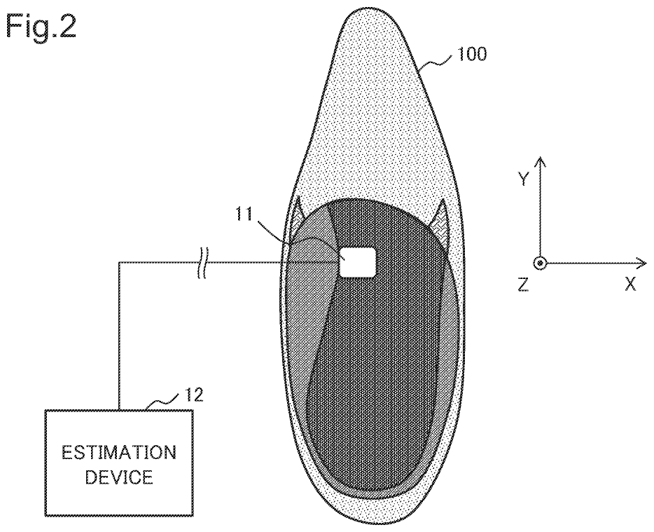

FIG. 2 is a conceptual diagram illustrating an example of the measurement device 11 disposed in a shoe 100. In the example of FIG. 2, the measurement device 11 is installed in the arrangement relevant to the back side of the arch of foot. For example, the measurement device 11 is disposed in an insole inserted into the shoe 100. For example, the measurement device 11 is disposed on the bottom surface of the shoe 100. For example, the measurement device 11 is embedded in the main body of the shoe 100. The measurement device 11 may be detachable from the shoe 100 or may not be detachable from the shoe 100. The measurement device 11 may be disposed at a position other than the back side of the arch of foot as long as the sensor data regarding the movement of the foot can be acquired. The measurement device 11 may be installed on a sock worn by the user or a decorative article such as an anklet worn by the user. The measurement device 11 may be directly attached to the foot or may be embedded in the foot. FIG. 2 illustrates an example in which the measurement device 11 is installed on the shoe 100 on the right foot side, but the measurement device 11 may be installed on the shoe 100 on the left foot side. The measurement device 11 may be installed on the shoes 100 for both feet. If the measurement device 11 is installed on the shoe 100 for both feet, the physical condition can be estimated based on the movement of the foot for both feet.

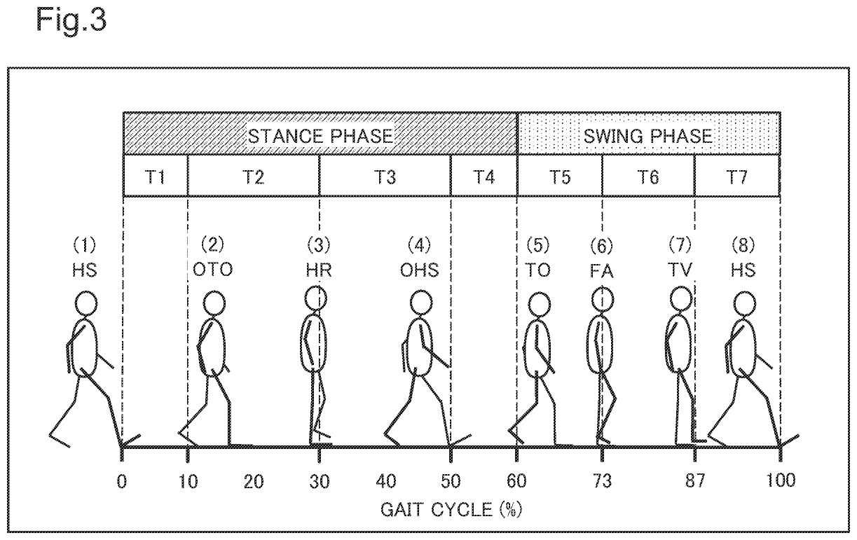

FIG. 3 is a conceptual diagram for explaining one gait cycle based on the right foot. The horizontal axis of FIG. 3 is a gait cycle normalized with one gait cycle of the right foot as 100%, with a time point at which the heel of the right foot lands on the ground as a start point and a time point at which the heel of the right foot next lands on the ground as an end point. The one gait cycle of one foot is roughly divided into a stance phase in which at least a part of the back side of the foot is in contact with the ground and a swing phase in which the back side of the foot is separated from the ground. In the present example embodiment, the normalization is performed such that the stance phase occupies 60% and the swing phase occupies 40%. The stance phase is further subdivided into an initial stance period T1, a mid-stance period T2, a terminal stance period T3, and a pre-swing period T4. The swing phase is further subdivided into an initial swing period T5, a mid-swing period T6, and a terminal swing period T7. The walking waveform in one gait cycle may not have a time point when the heel lands on the ground as the start point.

FIG. 3(1) illustrates an event (heel strike) in which the heel of the right foot is grounded (HS: Heel Strike). FIG. 3(2) illustrates an event (opposite toe off) in which the toe of the left foot is separated from the ground while the sole of the right foot is in contact with the ground (OTO: Opposite Toe Off). FIG. 3(3) illustrates an event (heel rise) in which the heel of the right foot rises while the sole of the right foot is in contact with the ground (HR: Heel Rise). FIG. 3(4) is an event (opposite heel strike) in which the heel of the left foot is grounded (OHS: Opposite Heel Strike). FIG. 3(5) illustrates an event (toe off) in which the toe of the right foot is separated from the ground while the sole of the left foot is in contact with the ground (TO: Toe Off). FIG. 3(6) illustrates an event (foot adjacent) in which the left foot and the right foot cross each other while the sole of the left foot is in contact with the ground (FA: Foot Adjacent). FIG. 3(7) illustrates an event (tibia vertical) in which the tibia of the right foot is substantially perpendicular to the ground while the sole of the left foot is in contact with the ground (TV: Tibia Vertical). FIG. 3(8) illustrates an event (heel strike) in which the heel of the right foot is grounded (HS: Heel Strike). FIG. 3(8) is relevant to the end point of the gait cycle starting from FIG. 3(1), and relevant to the start point of the next gait cycle.

The estimation device 12 acquires sensor data relevant to the walking of the subject from the measurement device 11 worn by the subject. The estimation device 12 acquires physical data such as the age of the subject input via an input device or the like (not illustrated). For example, physical data such as age, gender, and height is input to the estimation device 12. For example, the function of the estimation device 12 is installed in a mobile terminal (not illustrated) such as a smartphone or a tablet carried by the user. For example, the function of the estimation device 12 may be implemented in a server or a cloud.

The estimation device 12 estimates a probability (also referred to as a frailty probability) that the user is frail based on the sensor data acquired from the measurement device 11 worn by the user and the physical data of the user. Frailty indicates aging of the body and mind due to aging. In other words, the estimation device 12 estimates the frailty probability, which is one of the care-related information, using the sensor data (gait data) related to the movement of the foot.

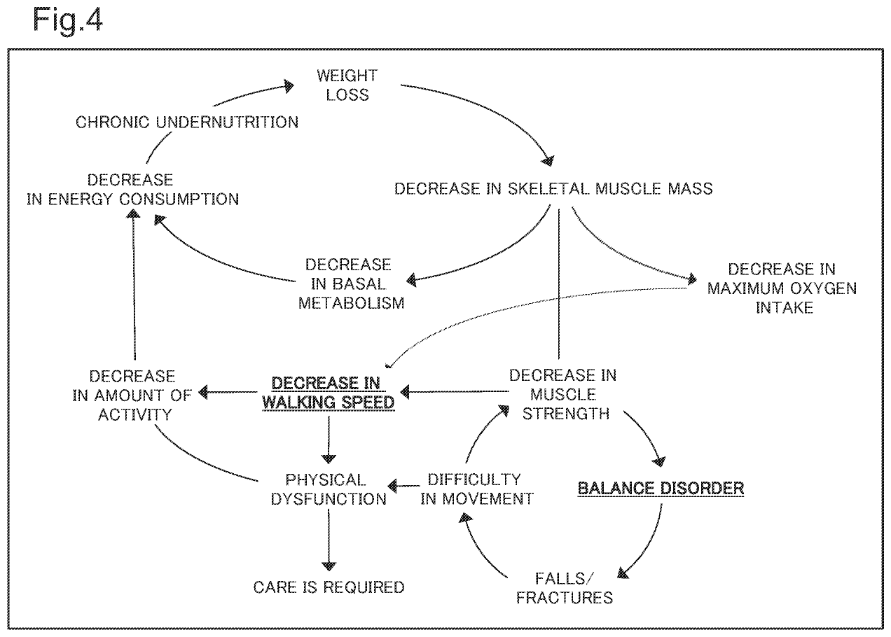

FIG. 4 is a conceptual diagram illustrating an example of a cycle of frailty (also referred to as a frail cycle). The frail cycle of FIG. 4 is based on FIG. 1 of NPL 1 (Q. Xue. et al., “Initial Manifestations of Frailty Criteria and the Development of Frailty Phenotype in the Women's Health and Aging Study II”, Journal of Gerontology: MEDICAL SCIENCES, 2008, 63A(9), pp. 984-990).

As in FIG. 4, frailty may progress with many factors intertwined. Decrease in energy consumption can lead to chronic undernutrition and contribute to weight loss. Weight loss decreases skeletal muscle mass, and can be a factor of basal metabolism, a decrease in maximum oxygen intake, and a decrease in muscle strength. A decrease in maximum oxygen intake may be a factor of a decrease in walking speed. A decrease in the walking speed may cause a decrease in the amount of activity or a physical dysfunction. When the amount of activity decreases, the energy consumption decreases. A decrease in muscle strength may cause a decrease in walking speed or a balance disorder. The balance disorder can be a factor in falls/fractures. When movement becomes difficult due to falls/fractures, the physical dysfunction is likely to occur. When the physical dysfunction progresses, there is a high possibility that care is required. In this way, many factors are intertwined with the physical condition. Therefore, it is required to avoid or delay the need for nursing care by detecting a sign of deterioration in physical condition.

In the present example embodiment, attention is paid to the walking speed in order to prevent the occurrence of the physical dysfunction and the need for nursing care. The walking speed can be an index of muscle strength and cardiopulmonary function. By improving/maintaining the muscle strength of the legs/hips, frailty can be prevented and its progression can be suppressed. In particular, in the present example embodiment, the frailty probability is estimated by focusing on the walking speed.

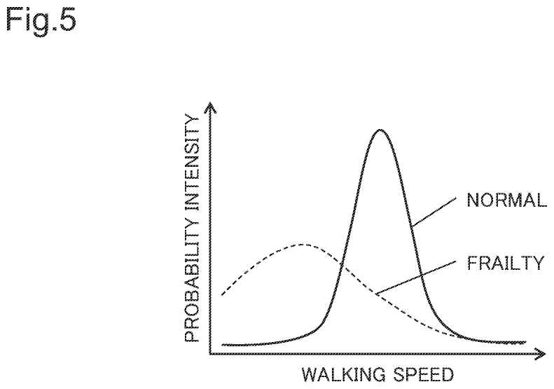

FIG. 5 is a graph for explaining the influence of frailty on the walking speed. The graph of FIG. 5 is based on the numerical values in Table 4 of NPL 2 (NPL 2: M. Schwenk, et al., “Wearable Sensor-Based In-Home Assessment of Gait, Balance, and Physical Activity for Discrimination of Frailty Status: Baseline Results of the Arizona Frailty Cohort Study”, Gerontology, 2015, 61(3), pp. 258-67). FIG. 5 illustrates a walking speed distribution relevant to a value of a walking speed (standard deviation) in single task walking in Table 4 of NPL 2. For a normal subject, the mean value of the walking speed is 1.17 meters/second (m/s) and the standard deviation is 0.15 m/s. For subjects with frailty symptoms, the mean value of the walking speed is 0.71 meters/second (m/s) with a standard deviation of 0.36 m/s. In FIG. 5, the walking speed of a normal subject is indicated by a solid line, and the walking speed of a subject having a frailty symptom is indicated by a broken line. As illustrated in FIG. 5, a subject having a frailty symptom tends to have a slow walking speed and a large distribution of walking speeds as compared with a normal subject.

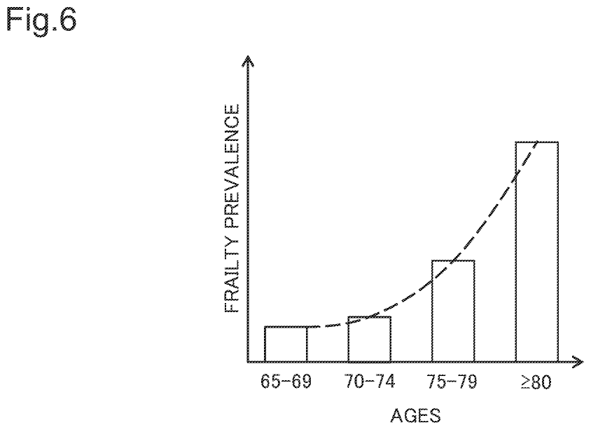

FIG. 6 is a frequency distribution showing the prevalence of frailty according to age. The frequency distribution of FIG. 6 is based on the numerical values in Table 1 of NPL 3 (NPL 3: H. Shimada, et al., “Combined Prevalence of Frailty and Mild Cognitive Impairment in a Population of Elderly Japanese People”, Journal of the American Medical Directors Association, 2013, pp. 1-7). FIG. 6 shows a curve (broken line) in which the frequency of the prevalence at the median value of each age group is smoothly connected. The dashed curve shows the correlation between age and frailty prevalence.

Here, an example in which the estimation device 12 estimates a probability that the user is frail (also referred to as a frailty probability) based on the walking speed will be described. In the following description, it is assumed that the frequency of occurrence of frailty depends on age, and the frailty affects the walking speed. The following Expression 1-1 represents a probability model in a case where it is assumed that the frequency of occurrence of the frailty f depends on the age y and the frailty f affects the walking speed v. The frailty f is 1 in the case of frailty, and is 0 in the case of non-frailty.

$$p ( y , f , v ) = p ( v \vert f ) \times p ( f \vert y ) \times p ( y ) \qquad ( 1 - 1 )$$

The first term p(v|f) on the right side of the above Expression 1-1 is a term relating to a frailty/non-frailty walking speed distribution. The second term p(f|y) on the right side is a term relating to the frailty prevalence. The third term p(y) is a term relating to age.

The following Expression 1-2 is an estimation Expression of the frailty probability p(f|y, v) according to the age y and the walking speed v based on the above Expression 1-1. The frailty probability p(f|y, v) is an example of the care-related information.

$$p ( f \vert y , v ) = \frac{p ( v \vert f ) \times p ( f \vert y )}{p ( v \vert y )} \qquad ( 1 - 2 )$$

The first term p(v|f) of the numerator on the right side of the above Expression 1-2 is a term relating to the frailty dependency of the walking speed distribution. The second term p(f|y) of the numerator is the age-dependent term for the incidence of frailty. The denominator p(v|y) is an age-dependent term of the walking speed. The estimation device 12 estimates the trail f according to the age y and the walking speed v based on Expression 1-2.

The following Expression 1-3 is an expression that embodies the first term p(v|f) of the numerator on the right side of the above Expression 1-2.

$$p ( v \vert f = i ) = \frac{1}{\sqrt{2 π} {σ}_{i}^{2}} exp ( - \frac{{( v - {μ}_{i}^{2} )}^{2}}{2 {σ}_{i}^{2}} ) \qquad ( 1 - 3 )$$

In Expression 1-3 above, i represents either non-frailty or frailty. μᵢ is the mean value of the walking speed with respect to i. σᵢ is the standard deviation of the walking speed with respect to i. Referring to Table 4 of NPL 2, when i is a non-frailty, μᵢ is 1.17 m/s and σᵢ is 0.15 m/s. Similarly, referring to Table 4 of NPL 2, when i is frailty, μᵢ is 0.71 m/s and σᵢ is 0.36 m/s.

The numerical value related to the frailty prevalence in NPL 3 can be applied to the second term p(f|y) of the numerator on the right side of Expression 1-2. The following Expression 1-4 is an equation obtained by approximating the curve (broken line) relating to the frailty prevalence in FIG. 6 with a sigmoid function.

$$p ( f \vert y ) = \frac{1}{1 + {e}^{- ( ay + b )}} \qquad ( 1 - 4 )$$

In the above Expression 1-4, the index of the second term of the denominator is “−(ay+b)” to include the influence of age y (a and b are real numbers).

When the above Expression 1-4 is modified, the following Expression 1-5 is obtained.

$$log ( \frac{p ( f \vert y )}{1 - p ( f \vert y )} ) = ay + b ≡ z ( y ) \qquad ( 1 - 5 )$$

By substituting age y and prevalence p(f|y) into Expression 1-5 above, a and b can be derived. In Table 1 of NPL 2, when the median value of each age group is age y, p(f|67.5) is 0.056, p(f|72.5) is 0.072, p(f|77.5) is 0.16, and p(f|85.0) is 0.349.

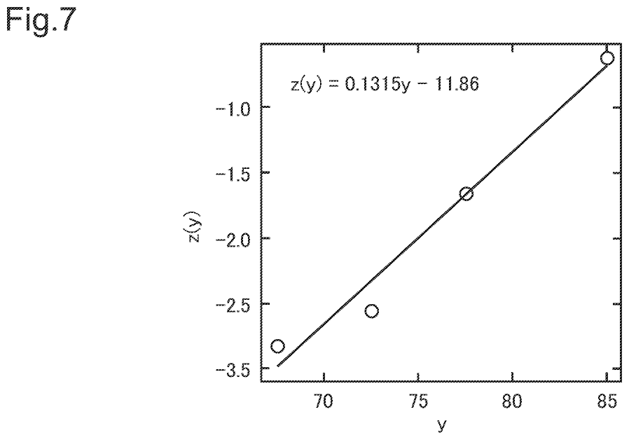

FIG. 7 is a graph obtained by plotting Z(y) against age y by substituting the above values into Expression 1-4. When the plotted points are linearly regressed by the least squares method, a is derived as 0.1315 and b is derived as −11.86. When the derived values of a and b are substituted into Expression 1-4, the following Expression 1-6 is obtained.

$$p ( f \vert y ) = \frac{1}{1 + {e}^{- ( 0.1315 y - 11.86 )}} \qquad ( 1 - 6 )$$

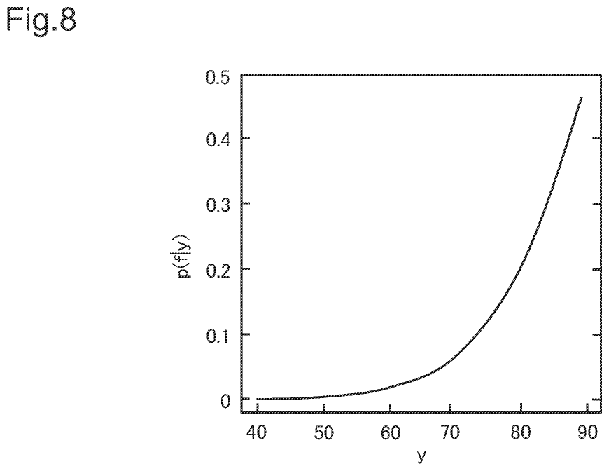

FIG. 8 is a graph relating to Expression 1-6 derived by the above procedure. By using the relationship (Expression 1-6) in the graph of FIG. 8, the frailty prevalence p(f|y) according to age y can be estimated. For example, physical data such as age y is input via an input device (not shown).

The estimation device 12 calculates the walking speed v relevant to the stride length per unit time based on the time-series data of the sensor data measured by the measurement device 11. A specific method for calculating the walking speed v will be described later. The estimation device 12 calculates a mean value μ and a standard deviation σ of the walking speeds v for a predetermined gait cycle. The estimation device 12 calculates the denominator p(v|y) of Expression 1-2 based on the age y of the user and the calculated mean value μ or standard deviation σ.

The estimation device 12 applies the age y of the user, the calculated walking speed v, and the like to the above Expression 1-2 to estimate the probability (frailty probability) that the user is frail. For example, the estimation device 12 stores an estimation model that outputs the frailty probability according to the inputs of the numerical value related to the walking speed v and the age y. For example, the estimation model can be generated by machine learning with the age y, the walking speed v, and the presence or absence of frailty as training data regarding a plurality of subjects.

The estimation device 12 outputs information regarding the estimated frailty probability. There is no particular limitation on the output destination of the information regarding the frailty probability. For example, the estimation device 12 outputs information regarding the user's frailty probability to an external system or device (not illustrated). For example, the estimation device 12 outputs information regarding the user's frailty probability to a display device (not illustrated).

The estimation device 12 may calculate a frail age as the care-related information. The frail age is the age according to the measure of the frailty estimated using the gait parameters and the physical data. The estimation device 12 calculates a frail age Y_{f} based on the following Expression 1-7.

$$p ( f \vert y , v ) = p ( f \vert {Y}_{f} ) \qquad ( 1 - 7 )$$

The left side p(f|y, v) of the above Expression 1-7 is a frailty probability according to the age y and the walking speed v. The right side p(f|Y_{f}) of Expression 1-7 is a frailty probability according to the frail age Y_{f}. The right side p(f|Y_{f}) of Expression 1-7 is a function of a curve relating to the age and the frailty prevalence indicated by a broken line in the frequency distribution of FIG. 6.

For example, the estimation device 12 applies the value obtained by the above Expression 1-2 to the left side p(f|y, v) of Expression 1-7. The estimation device 12 calculates a frail age Y_{f} that is the same as the value on the left side with respect to the function of the curve indicated by the broken line in FIG. 6. In other words, the estimation device 12 estimates the frail age of the user based on the estimated frailty probability and the correlation between the age and the frailty prevalence. For example, the estimation device 12 outputs the calculated information regarding the frail age as the care-related information.

### [Measurement Device]

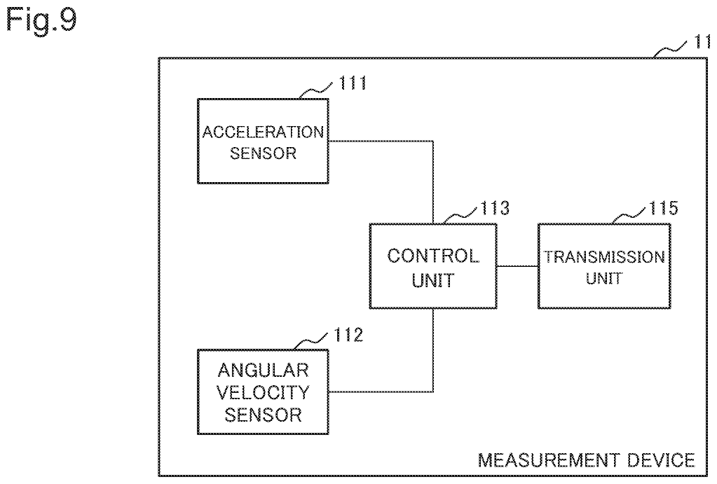

Next, details of the measurement device 11 will be described with reference to the drawings. FIG. 9 is a block diagram illustrating an example of a detailed configuration of the measurement device 11. The measurement device 11 includes an acceleration sensor 111, an angular velocity sensor 112, a control unit 113, and a transmission unit 115. The measurement device 11 includes a power supply (not illustrated).

The acceleration sensor 111 is a sensor that measures accelerations (also referred to as spatial accelerations) in three axial directions. The acceleration sensor 111 outputs the measured acceleration to the control unit 113. For example, a sensor of a piezoelectric type, a piezoresistive type, a capacitance type, or the like can be used as the acceleration sensor 111. The sensor used for the acceleration sensor 111 is not limited to the measurement method as long as the sensor can measure acceleration.

The angular velocity sensor 112 is a sensor that measures an angular velocity (also referred to as a spatial angular velocity) around three axes. The angular velocity sensor 112 outputs the measured angular velocity to the control unit 113. For example, a sensor of a vibration type, a capacitance type, or the like can be used as the angular velocity sensor 112. The sensor used for the angular velocity sensor 112 is not limited to the measurement method as long as the sensor can measure the angular velocity.

The control unit 113 acquires actual measurement values of accelerations in three axial directions from the acceleration sensor 111. The control unit 113 acquires actual measurement values of angular velocities around axes from the angular velocity sensor 112. The control unit 113 converts the acquired actual measurement values of the accelerations and the angular velocities into digital data (also referred to as sensor data). The control unit 113 outputs the converted digital data to the transmission unit 115. The sensor data includes at least acceleration data (including acceleration vectors in three axial directions) and angular velocity data (including angular velocity vectors around three axes) converted into digital data. The sensor data includes an acquisition time of an actual measurement value on which the acceleration data and the angular velocity data are based. The control unit 113 may be configured to output sensor data obtained by adding correction such as a mounting error, temperature correction, and linearity correction to the acquired acceleration data and angular velocity data. The control unit 113 may convert the coordinate system of the sensor data from the local coordinate system to the world coordinate system. The control unit 113 may generate angle data (also referred to as a plantar angle) around three axes using the acquired acceleration data and angular velocity data.

For example, the control unit 113 is a microcomputer or a microcontroller that controls the overall measurement device 11 or processes data. For example, the control unit 113 includes a central processing unit (CPU), a random access memory (RAM), a read only memory (ROM), a flash memory, and the like. The control unit 113 controls the acceleration sensor 111 and the angular velocity sensor 112 to measure the angular velocity and the acceleration. For example, the control unit 113 performs analog-to-digital conversion (AD conversion) on physical quantities (analog data) such as the measured angular velocity and acceleration, and stores the converted digital data in the flash memory. The physical quantity (analog data) measured by the acceleration sensor 111 and the angular velocity sensor 112 may be converted into digital data in each of the acceleration sensor 111 and the angular velocity sensor 112. The digital data stored in the flash memory is output to the transmission unit 115 at a predetermined timing.

The transmission unit 115 acquires sensor data from the control unit 113. The transmission unit 115 transmits the acquired sensor data to a measurement device 15. For example, the transmission unit 115 transmits the sensor data to the measurement device 15 via a wire such as a cable. For example, the transmission unit 115 transmits the sensor data to the measurement device 15 via wireless communication. For example, the transmission unit 115 is configured to transmit the sensor data to the measurement device 15 via a wireless communication function (not illustrated) conforming to a standard such as Bluetooth (registered trademark) or WiFi (registered trademark). The communication function of the transmission unit 115 may conform to a standard other than Bluetooth (registered trademark) or WiFi (registered trademark).

### [Estimation Device]

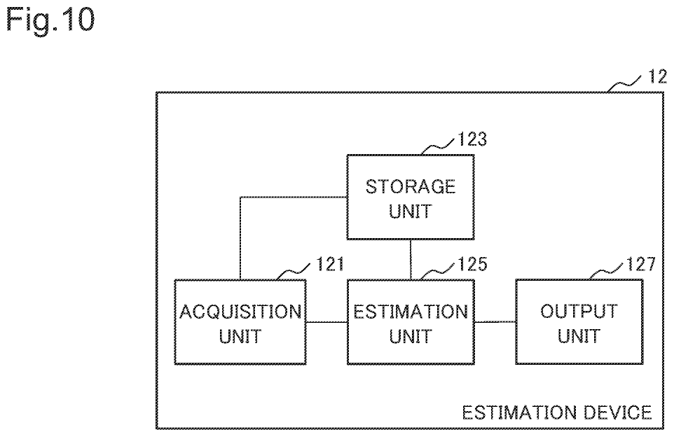

Next, details of the estimation device 12 will be described with reference to the drawings. FIG. 10 is a block diagram illustrating an example of the detailed configuration of the estimation device 12. The estimation device 12 includes an acquisition unit 121, a storage unit 123, an estimation unit 125, and an output unit 127.

The acquisition unit 121 acquires data (also referred to as physical data) such as the age of the user input via an input device (not illustrated). The acquisition unit 121 stores the acquired physical data in the storage unit 123. For example, the acquisition unit 121 receives the physical data from the input device via a wire such as a cable. For example, the acquisition unit 121 receives the physical data from the input device via wireless communication.

The acquisition unit 121 receives sensor data from the measurement device 11. The acquisition unit 121 outputs the received sensor data to the estimation unit 125. For example, the acquisition unit 121 receives the sensor data from the measurement device 11 via a wire such as a cable. For example, the acquisition unit 121 receives sensor data from the measurement device 11 via wireless communication.

For example, the acquisition unit 121 is connected to the measurement device 11 or the input device (not illustrated) via a wireless communication function (not illustrated) conforming to a standard such as Bluetooth (registered trademark) or WiFi (registered trademark). The communication function of the acquisition unit 121 may conform to a standard other than Bluetooth (registered trademark) or WiFi (registered trademark).

The storage unit 123 stores an estimation model for estimating the frailty probability of the subject based on the age and the walking speed distribution of the subject. The estimation model is constructed based on past knowledge. For example, the estimation model is a model in which Expression 1-3 or Expression 1-6 is applied to Expression 1-2 described above. The estimation model outputs the frailty probability according to a numerical value related to the walking speed distribution calculated using the sensor data measured according to the inputs of the walking of the user and the age of the user. The storage unit 123 stores the physical data of the user. The physical data includes at least the age of the subject. The physical data may include data such as gender, height, and weight of the user.

For example, the storage unit 123 may store an estimation model for estimating the user's frail age based on the user's age and walking speed distribution. For example, the estimation model is a model to which Expression 1-7 described above is applied. The estimation model outputs the frail age according to a numerical value related to the walking speed distribution calculated using the sensor data measured according to the inputs of the walking of the subject and the age of the subject.

The estimation unit 125 acquires the physical data on the subject from the storage unit 123. The estimation unit 125 acquires, from the acquisition unit 121, sensor data measured by the measurement device 11 installed on the footwear worn by the subject. The estimation unit 125 generates time-series data of the sensor data. The estimation unit 125 extracts walking waveform data for at least one gait cycle from the generated time-series data. The estimation unit 125 converts the coordinate system of the acquired sensor data from the local coordinate system to the world coordinate system. When the user is standing upright, the local coordinate system (x axis, y axis, z axis) and the world coordinate system (X axis, Y axis, Z axis) coincide. Since the spatial posture of the measurement device 11 changes while the user is walking, the local coordinate system (x axis, y axis, z axis) and the world coordinate system (X axis, Y axis, Z axis) do not match. Therefore, the estimation unit 125 converts the sensor data acquired by the measurement device 11 from the local coordinate system (x axis, y axis, z axis) of the measurement device 11 into the world coordinate system (X axis, Y axis, Z axis). The estimation unit 125 generates time-series data (also referred to as a walking waveform) of the sensor data after conversion into the world coordinate system.

For example, the estimation unit 125 generates time-series data such as a spatial acceleration and a spatial angular velocity. For example, the estimation unit 125 integrates the spatial acceleration and the spatial angular velocity to generate time-series data such as a spatial speed, a spatial angle (also referred to as a plantar angle). For example, the estimation unit 125 performs second-order integration on the spatial acceleration and the spatial angular velocity to generate time-series data such as a spatial trajectory. The estimation unit 125 generates time-series data at a predetermined timing or time interval set in accordance with a general gait cycle or a gait cycle unique to the user. The timing at which the estimation unit 125 generates the time-series data can be arbitrarily set. For example, the estimation unit 125 is configured to continue to generate time-series data during a period in which the user keeps walking. The estimation unit 125 may be configured to generate time-series data at a specific time.

The estimation unit 125 detects a walking event from the generated walking waveform. For example, the estimation unit 125 extracts a feature specific to the walking event from the walking waveform. For example, the estimation unit 125 detects the timing of the extracted feature for the walking event as the timing of the walking event. For example, the estimation unit 125 detects the toe off or the heel strike as the walking event. For example, the estimation unit 125 detects the foot adjacent as the walking event. The foot adjacent corresponds to a timing at which the toe of the right foot passes the position of the midpoint between the toe and the heel of the left foot when the right foot is used as a reference. For example, the estimation unit 125 may detect a tibial vertical, an opposite toe off, and an opposite heel strike as the walking event.

The estimation unit 125 calculates the stride length of the subject based on the detected walking event. For example, the estimation unit 125 calculates the walking speed of the subject by dividing the stride length by the time relevant to one stride.

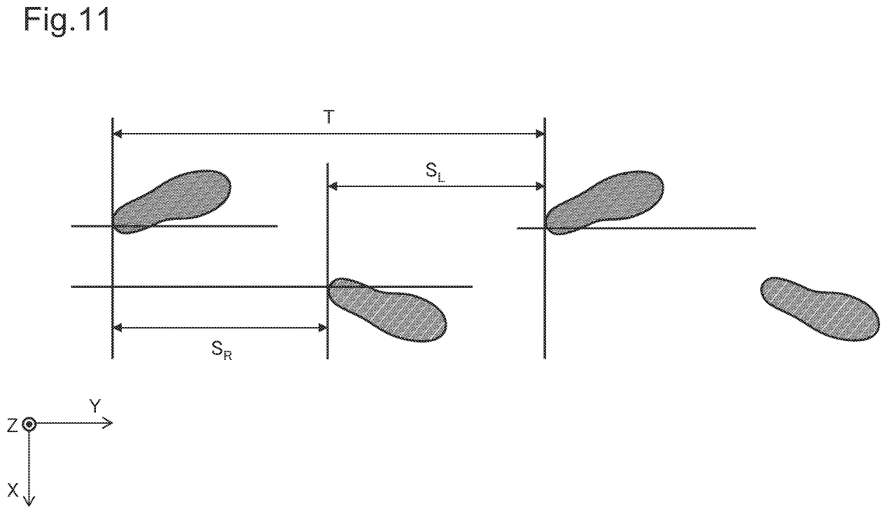

FIG. 11 is a conceptual diagram for explaining gait parameters such as a step length and a stride length. In FIG. 11, regarding the walking subject, the right direction is X, the traveling direction is Y, and the vertical upward direction is X. FIG. 11 illustrates a right-foot step length S_{R}, a left-foot step length S_{L}, and a stride length T. The right-foot step length S_{R} is a difference in the Y-coordinate between the heel of the right foot and the heel of the left foot when the state in which the sole of the left foot is in contact with the ground transitions to the state in which the heel of the right foot swung out in the traveling direction lands. The left-foot step length S_{L} is a difference between the Y-coordinates of the heel of the left foot and the heel of the right foot when the state in which the sole of the right foot is in contact with the ground transitions to the state in which the heel of the left foot swung out in the traveling direction lands. The stride length T is relevant to the sum of the right-foot step length S_{R} and the left-foot step length S_{L}. However, the right-foot step length S_{R} and the left-foot step length S_{L} may have different values depending on the walking event used for the calculation.

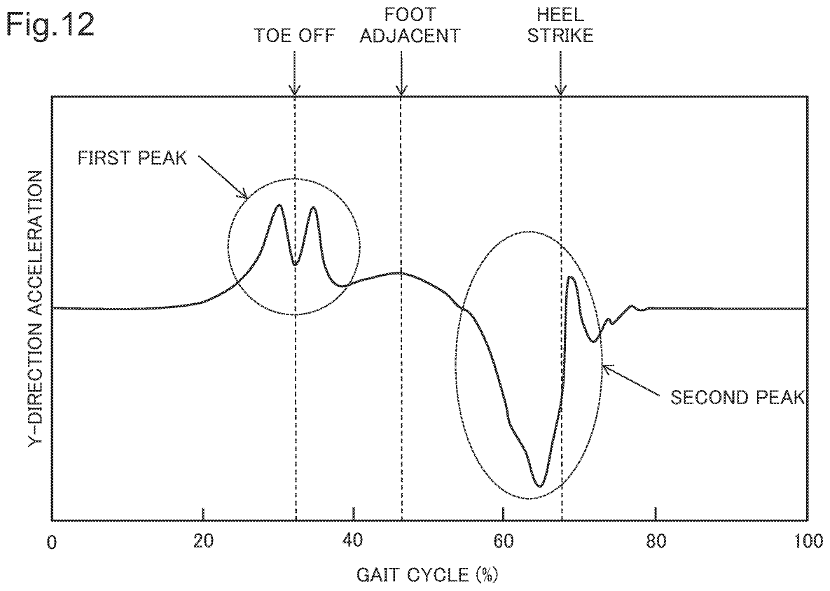

FIG. 12 is an example of the walking waveform measured by the measurement device 11. FIG. 12 is an example of a walking waveform of the Y-direction acceleration for one gait cycle with the central timing of the stance phase (start of the terminal stance period) as the start point. In the walking waveform of the Y-direction acceleration for one gait cycle, two main peaks (first peak, second peak) appear. The first peak appears around 20 to 40% of the gait cycle. The first peak includes two maximum peaks and one minimum peak. The timing of the minimum peak included in the first peak corresponds to the timing of the toe off. The second peak appears around 50 to 70% of the gait cycle. The second peak includes a minimum peak around a gait cycle exceeding 60% and a maximum peak around a gait cycle of 70%. The timing of the midpoint between the minimum peak and the maximum peak included in the second peak is relevant to the timing of the heel strike. The maximum timing of the gentle peak between the first peak and the second peak is relevant to the timing of the foot adjacent.

For example, the estimation unit 125 calculates the stride length T using a walking waveform (not illustrated) of a Y-direction trajectory for one gait cycle. The Y-direction trajectory is obtained by second-order integration of Y-direction acceleration for one gait cycle. The estimation unit 125 calculates a difference between the spatial position in the Y direction at the timing of the heel strike and the spatial position in the Y direction at the timing of the toe off as the stride length T. The estimation unit 125 calculates the time from the timing of the toe off to the timing of the heel strike as the time t of one stride. The estimation unit 125 calculates the walking speed v by dividing the stride length T by the time t of one stride. For example, the estimation unit 125 can calculate a moving distance in a period from the toe off to the foot adjacent or a moving distance in a period from the foot adjacent to the heel strike as the step length.

The estimation unit 125 calculates a mean value μ and a standard deviation σ of the walking speeds v for a predetermined gait cycle. For example, the estimation unit 125 calculates the mean value μ and the standard deviation σ of the walking speed v using the sensor data for 3 to 10 gait cycles. The estimation unit 125 calculates the mean value μ and the standard deviation σ as numerical values related to the walking speed distribution.

The estimation unit 125 inputs the physical data (age) of the subject and the numerical value related to the walking speed distribution calculated based on the walking of the subject to the estimation model for estimating the frailty probability stored in the storage unit 123. The estimation unit 125 outputs the frailty probability output from the estimation model to the output unit 127 as the care-related information according to the inputs of the numerical value related to the walking speed distribution and the physical data.

For example, the estimation unit 125 may estimate the frail age of the subject based on the age and the walking speed distribution of the subject. The estimation unit 125 inputs the physical data (age) of the user and the numerical value related to the walking speed distribution calculated based on the walking of the user to the estimation model for estimating the frail age stored in the storage unit 123. The estimation unit 125 outputs the frail age output from the estimation model to the output unit 127 as the care-related information according to the inputs of the numerical value related to the walking speed distribution and the physical data.

The output unit 127 outputs the care-related information estimated by the estimation unit 125. The output destination (not illustrated) of the care-related information is not particularly limited. For example, the output unit 127 outputs the care-related information to the output destination via a wire such as a cable. For example, the acquisition unit 121 outputs the care-related information to the output destination via wireless communication. For example, the output unit 127 is connected to an output destination via a wireless communication function (not illustrated) conforming to a standard such as Bluetooth (registered trademark) or WiFi (registered trademark). The communication function of the output unit 127 may conform to a standard other than Bluetooth (registered trademark) or WiFi (registered trademark).

For example, the output unit 127 outputs the care-related information of the user to an external system or device (not illustrated). For example, the output unit 127 outputs the care-related information to a display device (not illustrated). For example, the output unit 127 outputs the care-related information to a portable device (not illustrated) of the user. For example, the output unit 127 outputs the care-related information of the user to a terminal device or the like (not illustrated) that allows a doctor or the like who is examining the physical condition of the user to browse. The output care-related information can be used for any purpose.

### (Operation)

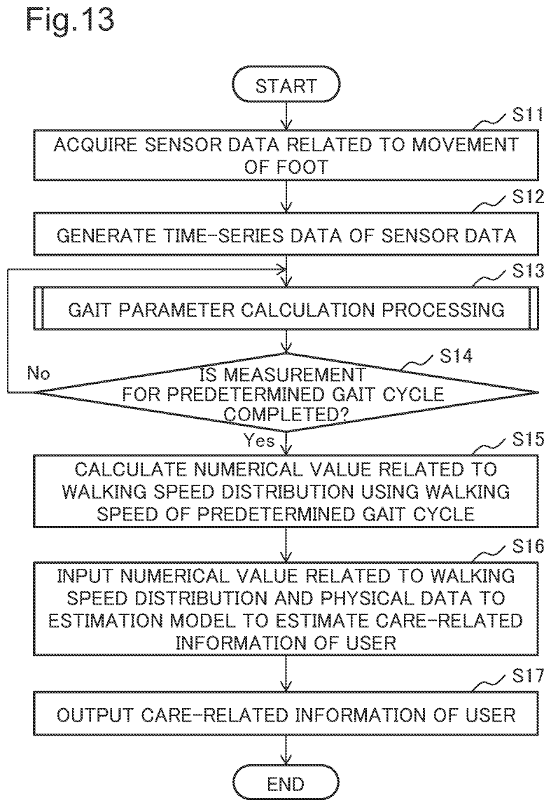

Next, an operation of the information presentation system 1 will be described with reference to the drawings. Hereinafter, the operation of the measurement device 11 will not be described, and the operation of the estimation device 12 will be described. FIG. 13 is a flowchart for explaining an example of the operation of the estimation device 12. In the description along the flowchart of FIG. 13, the estimation device 12 will be described as an operation subject.

In the flowchart of FIG. 13, first, the estimation device 12 acquires sensor data regarding the movement of the foot (step S11). The estimation device 12 acquires sensor data regarding the movement of the foot from the measurement device 11. The estimation device 12 may acquire the sensor data measured by the measurement device 11 for each gait cycle, or may collectively acquire the sensor data for a plurality of gait cycles.

Next, the estimation device 12 generates time-series data of sensor data (step S12). The estimation device 12 generates time-series data of sensor data regarding accelerations and trajectories in three axis directions, angular velocities around three axes, and the like. In the present example embodiment, the estimation device 12 generates time-series data of sensor data regarding acceleration and a trajectory in the Y direction.

Next, the estimation device 12 executes gait parameter calculation processing (step S13). In the gait parameter calculation processing, the estimation device 12 calculates the walking speed based on the time-series data (walking waveform) of the sensor data for one gait cycle. Details of the gait parameter calculation processing will be described later.

When the calculation for the predetermined gait cycle is completed (Yes in step S14), the estimation device 12 derives a numerical value related to the walking speed distribution using the walking speed for the predetermined gait cycle (step S15). The numerical value related to the walking speed distribution includes a mean value and a standard deviation of the walking speed in walking for a predetermined gait cycle. When the calculation for the predetermined gait cycle is not completed (No in step S14), the process returns to step S13.

The estimation device 12 inputs the numerical value related to the walking speed distribution and the physical data to the estimation model to estimate the care-related information of the user (step S16). For example, the estimation device 12 estimates a frailty probability and a frail age as the care-related information.

The estimation device 12 outputs the care-related information of the user (step S17). For example, the estimation device 12 outputs the frailty probability and the frail age as the care-related information. There is no particular limitation on the output destination and use of the care-related information of the user.

### [Gait Parameter Calculation Processing]

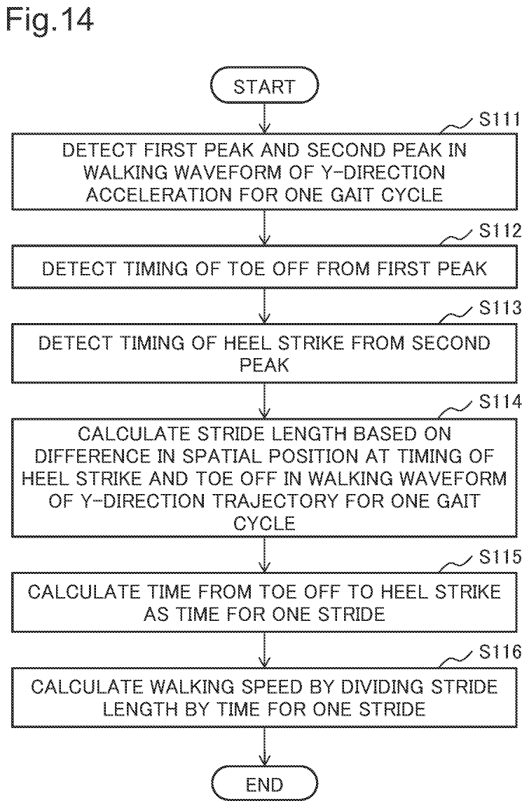

Next, gait parameter calculation processing by the estimation device 12 will be described with reference to the drawings. FIG. 14 is a flowchart for explaining an example of the gait parameter calculation processing. In the description along the flowchart of FIG. 14, the estimation device 12 will be described as an operation subject.

In FIG. 13, first, the estimation device 12 detects the first peak and the second peak in the walking waveform of the Y-direction acceleration (traveling direction acceleration) for one gait cycle (step S111).

Next, the estimation device 12 detects the timing of the toe off from the first peak (step S112).

Next, the estimation device 12 detects the timing of heel strike from the second peak (step S113).

Next, the estimation device 12 calculates the stride length based on the difference in the spatial position at the timing of the heel strike and the toe off in the walking waveform of the Y-direction trajectory (traveling direction trajectory) for one gait cycle (step S114).

Next, the estimation device 12 calculates the time from the toe off to the heel strike as the time for one stride (step S115).

Next, the estimation device 12 calculates the walking speed by dividing the stride length by the time relevant to one stride (step S116).

### Application Example 1

Next, Application Example 1 of the information presentation system 1 of the present example embodiment will be described with reference to the drawings. In the present application example, information regarding the frailty probability estimated by the estimation device 12 is displayed on the screen of a mobile terminal 110. In the present application example, it is assumed that the measurement device 11 is installed in the shoe 100 of the user, and the sensor data based on the physical quantity related to the movement of the foot measured by the measurement device 11 is transmitted to the mobile terminal 110 possessed by the user. It is assumed that the sensor data transmitted to the mobile terminal 110 is subjected to data processing by a program (estimation device 12) installed in the mobile terminal 110.

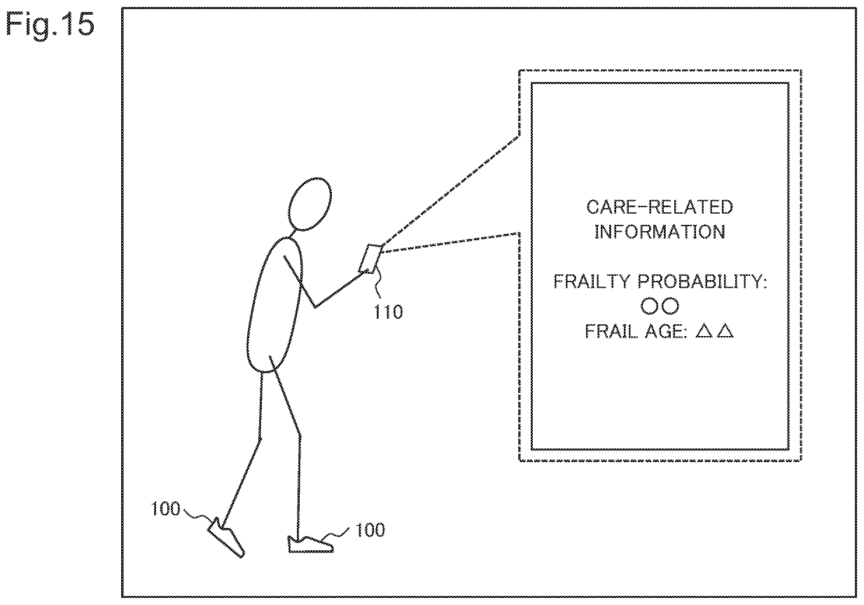

FIG. 15 is an example in which information regarding the physical condition of the user is displayed on the screen of the mobile terminal 110 of the user wearing the shoe 100 on which the measurement device 11 is installed. In the example of FIG. 15, the frailty probability and the frail age estimated based on the walking of the user are displayed on the screen of the mobile terminal 110 of the user as the care-related information. The user who has browsed the care-related information displayed on the screen of the mobile terminal 110 can take an action according to the information. For example, a pedestrian who has browsed the care-related information displayed on the screen of the mobile terminal 110 can consult a medical institution, a care-related facility, or the like according to the information.

For example, the information presentation system 1 may display, on the screen of the mobile terminal 110, recommendation information recommending consultation with an appropriate medical institution, care-associated facility, or the like, according to the estimated care-related information. For example, the information presentation system 1 may display, on the screen of the mobile terminal 110, recommendation information including a telephone number, an e-mail address, or the like of an appropriate medical institution, care-associated facility, or the like according to the care-related information. For example, the information presentation system 1 may present recommendation information for promoting an appropriate exercise according to the estimated care-related information. For example, the information presentation system 1 may present estimated information regarding physical balance and muscle strength according to the estimated care-related information. For example, the information presentation system 1 may present the temporal change of the accumulated past care-related information in a graph.

As described above, the information presentation system of the present example embodiment includes the measurement device and the estimation device. The measurement device is disposed on the user's footwear. The measurement device measures the spatial acceleration and the spatial angular velocity according to the gait of the user. The measurement device generates sensor data based on the measured spatial acceleration and spatial angular velocity. The measurement device outputs the generated sensor data to the estimation device, and the estimation device includes an acquisition unit, a storage unit, an estimation unit, and an output unit. The acquisition unit acquires sensor data measured according to walking of the user and physical data of the user. The storage unit stores the estimation model that outputs the care-related information according to the inputs of the feature quantity extracted from the sensor data and the physical data, and the physical data. The estimation unit inputs the feature quantity extracted from the sensor data of the user and the physical data to the estimation model to estimate the care-related information of the user. The output unit outputs the estimated care-related information of the user.

The information presentation system according to the present example embodiment uses an estimation model that outputs care-related information according to inputs of a feature quantity extracted from sensor data and physical data. Therefore, according to the information presentation system of the present example embodiment, it is possible to estimate the care-related information according to the physical condition of the user based on the gait of the user.

In one aspect of the present example embodiment, the storage unit stores an estimation model that outputs the care-related information according to the inputs of the numerical value related to the distribution of the walking speed and the physical data. The estimation unit generates a walking waveform for a predetermined gait cycle by using time-series data of sensor data for the predetermined gait cycle of the user. The estimation unit calculates a predetermined gait parameter for each gait cycle based on the walking event detected from the walking waveform for the predetermined gait cycle. The estimation unit calculates the walking speed of the user using the calculated predetermined gait parameter. The estimation unit calculates a numerical value related to the distribution of the walking speed measured according to the walking of the user in the predetermined gait cycle. The estimation unit inputs the numerical value related to the distribution of the calculated walking speeds and the physical data to the estimation model. The estimation unit estimates the care-related information of the user. According to the present aspect, it is possible to estimate the care-related information according to the physical condition of the user by inputting the numerical value related to the distribution of the walking speed and the physical data to the estimation model.

In one aspect of the present example embodiment, the estimation unit generates a walking waveform of the acceleration in the traveling direction and a walking waveform of the trajectory in the traveling direction by using sensor data for a predetermined gait cycle of the user. The estimation unit detects the toe off and the heel strike as the walking event from the walking waveform of the acceleration in the traveling direction. The estimation unit extracts a spatial position at the timing of the toe off and the heel strike in the walking waveform of the trajectory in the traveling direction. The estimation unit calculates, as the stride length of the user, a difference between the extracted spatial positions at the timing of the toe off and the heel strike. The estimation unit calculates the time from the toe off to the heel strike as the time of one stride. The estimation unit calculates the walking speed by dividing the time by the stride length for one stride. According to the present aspect, the walking speed can be calculated based on the timing of the toe off and the heel strike detected from the walking waveform of the acceleration in the traveling direction.

In one aspect of the present example embodiment, the acquisition unit acquires the age of the user as the physical data. The storage unit stores an estimation model that outputs a frailty probability as the care-related information according to the inputs of the numerical value related to the distribution of the walking speed and the age of the user. The estimation unit inputs a numerical value related to the distribution of the walking speed and the age of the user to the estimation model, and estimates the frailty probability of the user. According to the present aspect, the frailty probability of the user can be estimated based on the numerical value related to the distribution of the walking speed of the user and the age of the user.

In one aspect of the present example embodiment, the estimation unit estimates the frail age of the user based on the estimated frailty probability and the correlation between the age and the frailty prevalence. According to the present aspect, the frail age of the user can be estimated based on the frailty probability of the user.

In one aspect of the present example embodiment, the estimation unit outputs the care-related information to a terminal device having a screen, and displays the care-related information on the screen of the terminal device. According to the present aspect, the care-related information estimated based on the gait of the user can be displayed on the screen of the terminal device.

The information presentation system of the present example embodiment may be configured to present information regarding sarcopenia. Sarcopenia is a phenomenon in which a decrease in muscle mass due to aging or diseases causes a decrease in muscle strength of the entire body such as a grip strength, lower limb muscles, and trunk muscles. Sarcopenia also includes a decrease in physical function such as a decrease in walking speed due to a decrease in muscle strength of the entire body. For example, an estimation model for estimating the probability of sarcopenia (also referred to as a sarcopenia probability) is generated according to the inputs of the physical data and the numerical value related to the walking speed distribution. By using such an estimation model, the sarcopenia probability can be estimated similarly to the frailty. For example, the information presentation system of the present example embodiment may be configured to estimate a sarcopenia age, similar to the frail age. The sarcopenia age is the age according to the measure of sarcopenia estimated using the gait parameters and physical data.

The care-related information output by the information presentation system of the present example embodiment may be used not only as the estimation of the frailty and the sarcopenia but also as a determination criterion of the degree of required care and the degree of required support. For example, if an estimation model for estimating the degree of required care and the degree of required support is generated according to the inputs of the physical data and the numerical value related to the walking speed distribution, the degree of required care and the degree of required support can be estimated. For example, if an estimation model for estimating the reference time such as the recognition of required care is generated according to the inputs of the physical data and the numerical value related to the walking speed distribution, the degree of required care and the degree of required support can be estimated according to the reference time such as the recognition of required care.

### Second Example Embodiment

Next, an information presentation system according to a second example embodiment will be described with reference to the drawings. In addition to the walking speed, the information presentation system of the present example embodiment also focuses on the walking variation associated with balance, and estimates the falling easiness indicating the likelihood of falling.

### (Configuration)

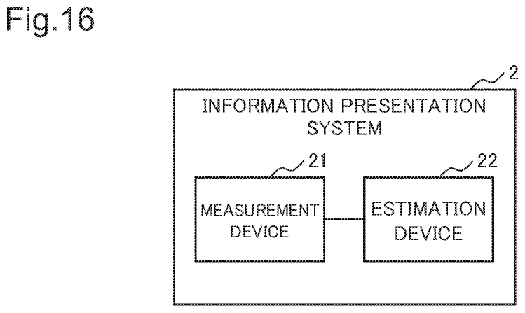

FIG. 16 is a block diagram illustrating a configuration of an information presentation system 2 according to the present example embodiment. The information presentation system 2 includes a measurement device 21 and an estimation device 22. The measurement device 21 and the estimation device 22 may be connected by wire or wirelessly. The measurement device 21 and the estimation device 22 may be configured by a single device. The information presentation system 2 may be configured only by the estimation device 22 excluding the measurement device 21 from the information presentation system 2. The measurement device 21 has a configuration similar to that of the measurement device 11 according to the first example embodiment. Therefore, in the present example embodiment, a detailed description of the measurement device 21 is omitted.

In the present example embodiment, attention is paid to the walking speed and balance in order to prevent the occurrence of the physical dysfunction and the need for nursing care. The walking speed can be an index of muscle strength and cardiopulmonary function. By maintaining the balance by improving/maintaining the muscle strength of the legs/hips, it is possible to prevent frailty and to suppress the progression thereof. In particular, in the present example embodiment, the likelihood of falling (falling easiness) is estimated by focusing on the walking speed and the walking variation.

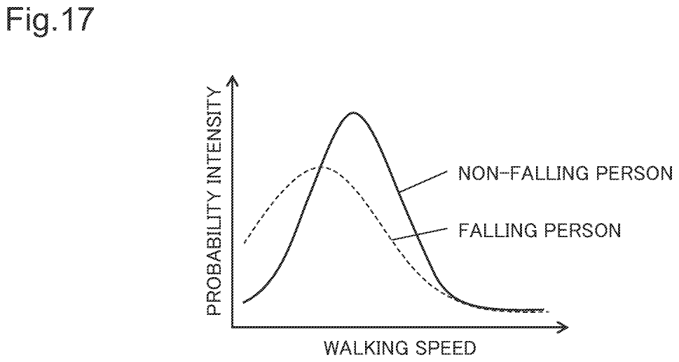

FIG. 17 is a graph for explaining the walking speeds of a subject having experience of falling (falling person) and a subject having no experience of falling (non-falling person) within a specific verification period. The graph of FIG. 17 is based on the numerical values in Table 3 of NPL 4 (NPL4: J. Hausdorff, et al., “Gait Variability and Fall Risk in Community-Living Older Adults: A 1-Year Prospective Study”, Arch Phys Med Rehabil, Vol 82, August 2001, pp. 1050-1056). FIG. 17 illustrates a walking speed distribution relevant to the value of the average walking speed (standard deviation) in Table 3 of NPL 4. For a person who does not fall, the mean value of the walking speed is 0.91 meters/second (m/s), and the standard deviation is 0.24 m/s. For a falling person, the mean value of the walking speed is 0.71 meters/second (m/s), and the standard deviation is 0.33 m/s. In FIG. 17, the walking speed of the non-falling person is indicated by a solid line, and the walking speed of the falling person is indicated by a broken line. As illustrated in FIG. 17, a falling person tends to have a slower walking speed and a larger distribution of walking speeds than a non-falling person.

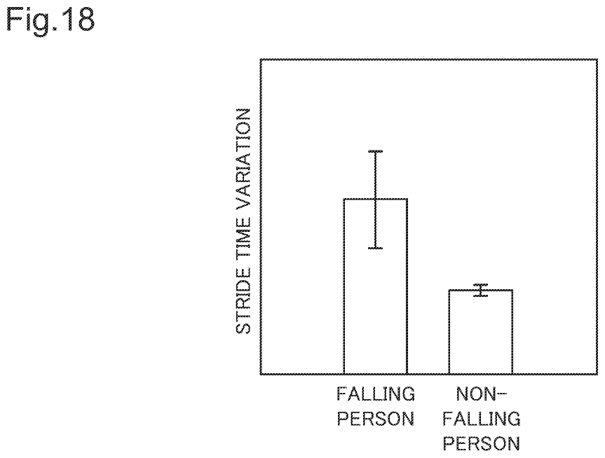

FIG. 18 is a graph for explaining stride time variations of a subject having experience of falling (falling person) and a subject having no experience of falling (non-falling person) within a specific period. The graph of FIG. 18 is based on FIG. 1 of NPL 4. The stride time is a time of a stance phase in one gait cycle. The stride time variation is relevant to the variance in stride time. In the present example embodiment, the time from the heel strike to the toe off is set as the stride time. For example, the stride time is obtained by subtracting a time of one stride from a time of one gait cycle. As illustrated in FIG. 18, the falling person tends to have a large stride time variation and a large distribution of stride time variation as compared with the non-falling person.

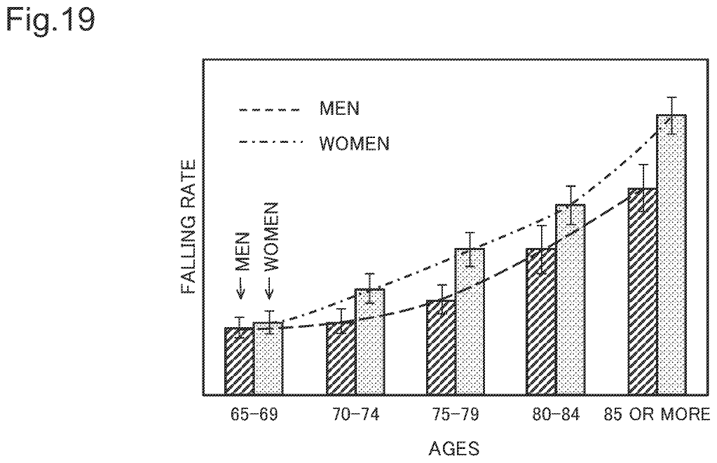

FIG. 19 is a frequency distribution showing the falling rate according to age. The frequency distribution of FIG. 19 is based on FIG. 3 of NPL 5 (C. Pearson, et al., “Understanding seniors' risk of falling and their perception of risk”, Statistics Canada, Catalogue no. 82-624-X, Health at a Glance, October 2014, pp. 1-11). FIG. 19 illustrates the falling rate for each of men and women. FIG. 19 illustrates a curve smoothly connecting the frequency of the falling rate at the median value of each age group. The dashed curve shows the correlation between age and falling rate for men. The dash-dot curve shows the correlation between age and falling rate for men.

Here, an example in which the estimation device 22 estimates the ease of falling (also referred to as falling easiness) of the user based on the walking speed and the walking variation will be described. In the following description, it is assumed that the falling easiness depends on age and gender. It is assumed that the falling easiness affects the walking speed and the walking variation. The following Expression 2-1 represents the probability of falling easiness in a case where it is assumed that a falling easiness fₐ depends on the age y and the gender s, and the falling easiness fₐ affects the walking speed v and the walking variation W.

$$p ( {f}_{a} \vert y , s , v , w ) = \frac{p ( v \vert {f}_{a} ) \times p ( w \vert {f}_{a} ) \times p ( {f}_{a} \vert y , s )}{p ( v , w \vert y , s )} \qquad ( 2 - 1 )$$

The first term p(v|fₐ) of the numerator on the right side of the above Expression 2-1 is a term relating to the walking speed distribution of the fallen person/non-fallen person. The second term p(w|fₐ) of the numerator is a term relating to the walking variation distribution of the fallen/non-fallen person. The third term p(fₐ|y, s) of the numerator is the term relating to the falling rate according to the age y and the gender s. The denominator p(v, w|y, s) is a term relating to the walking speed distribution and the walking variation distribution according to the age y and the gender s. For example, physical data such as the age y and the gender s may be input via an input device (not illustrated). The estimation device 22 estimates the falling easiness fₐ according to the age y, the gender s, the walking speed v, and the walking variation w based on Expression 2-1.

The following Expression 2-2 is an expression that embodies the first term p(v|fₐ) of the numerator on the right side of the above Expression 2-1.

$$p ( v \vert {f}_{a} = j ) = \frac{1}{\sqrt{2 π} {σ}_{1 j}^{2}} exp ( - \frac{{( v - {μ}_{1 j}^{2} )}^{2}}{2 {σ}_{1 j}^{2}} ) \qquad ( 2 - 2 )$$

In Expression 2-2 above, j represents either a non-falling person or a falling person. μ₁ⱼ is a mean value of the walking speed with respect to j. σ₁ⱼ is the standard deviation of the walking speed with respect to j. Based on Table 3 of NPL 4, when j is a person who does not fall, μ₁ⱼ is 0.91 m/s, and σ₁ⱼ is 0.24 m/s. Based on Table 3 of NPL 4, when j is a falling person, μ₁ⱼ is 0.71 m/s, and σ₁ⱼ is 0.33 m/s.

Expression 2-3 below embodies the second term p(w|fₐ) of the numerator on the right side of Expression 2-1 above.

$$p ( w \vert {f}_{a} = j ) = \frac{1}{\sqrt{2 π} {σ}_{2 j}^{2}} exp ( - \frac{{( v - {μ}_{2 j}^{2} )}^{2}}{2 {σ}_{2 j}^{2}} ) \qquad ( 2 - 3 )$$

In Expression 2-3 above, j represents either a non-falling person or a falling person. From the graph of the stride time variation of FIG. 18 (FIG. 1 of NPL 4), the walking variation distribution of the non-falling/falling person can be read. Based on FIG. 18, the stride time variation of the person who does not fall has a mean value of 50.3 milliseconds (ms) and a standard deviation of 4.2 ms. Based on FIG. 18, the stride time variation of the falling person has a mean value of 105.9 ms and a standard deviation of 30.1 ms.

The numerical value related to the falling rate for each age group/gender in FIG. 3 of NPL 5 can be applied to the third term p(fₐ|y, s) of the numerator on the right side of Expression 2-1. The following Expression 2-4 is an equation in which the curve (broken line) relating to the falling rate in FIG. 19 is approximated by a sigmoid function.

$$p ( {f}_{a} \vert y , s ) = \frac{1}{1 + {e}^{- ( {a}_{s} y + {b}_{s} )}} \qquad ( 2 - 4 )$$

In the above Expression 1-4, the index of the second term of the denominator is “−(aₛy+bₛ)” to include the influence of the age y (aₛ and bₛ are real numbers). aₛ and bₛ are constants according to gender.

When the above Expression 2-4 is transformed, the following Expression 2-5 is obtained.

$$log ( \frac{p ( {f}_{a} \vert y , s )}{1 - p ( {f}_{a} \vert y , s )} ) = {a}_{s} y + {b}_{s} ≡ {z}_{s} ( y ) \qquad ( 2 - 5 )$$

By substituting the age y and the falling rate p(fₐ|y, s) into Expression 2-5 above, aₛ and bₛ can be derived. Referring to FIG. 3 of NPL 5, when the median value of each age group is the age y, p(fₐ|67.5) is 0.122, p(fₐ|72.5) is 0.131, p(fₐ|77.5) is 0.171, p(fₐ|82.5) is 0.261, and p(fₐ|90) is 0.372 for men. Similarly, regarding women, p(fₐ|67.5) is 0.129, p(fₐ|72.5) is 0.190, p(fₐ|77.5) is 0.260, p(fₐ|82.5) is 0.340, and p(fₐ|90) is 0.500.

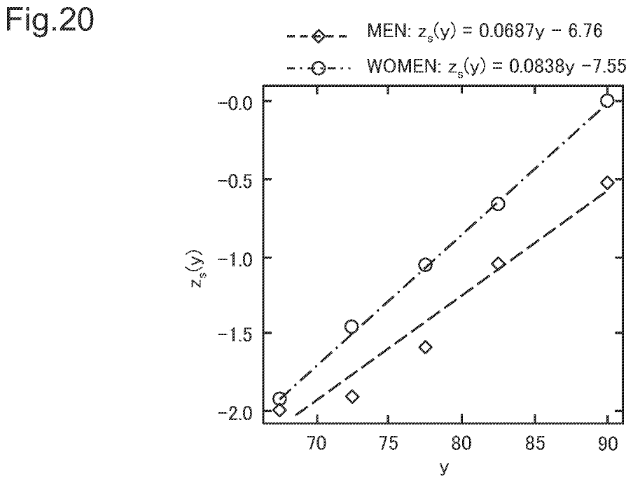

FIG. 20 is a graph obtained by plotting Zₛ(y) with respect to the age y by substituting the above values for each of men and women into Expression 2-5. Diamonds (⋄) are plots relating to men. A straight line obtained by linearly regressing the plot for men is indicated by a broken line. Circular (∘) is a plot for women. A straight line obtained by linearly regressing a plot relating to women is indicated by a dash-dot line. When the points plotted with respect to men are linearly regressed by the least squares method, aₛ is derived as 0.0687 and bₛ is derived as −6.76. When the points plotted with respect to female are linearly regressed by the least squares method, aₛ is derived as 0.0838 and bₛ derived as −7.55. Substituting the derived values of aₛ and bₛ into Expression 2-4 for each of man and woman results in Expression 2-6 and Expression 2-7 below.

$$p ( {f}_{a} \vert y , m ) = \frac{1}{1 + {e}^{- ( 0.0687 y - 6.76 )}} \qquad ( 2 - 6 )$$

$$p ( {f}_{a} \vert y , f ) = \frac{1}{1 + {e}^{- ( 0.0838 y - 7.55 )}} \qquad ( 2 - 7 )$$

Expression 2-6 above shows the falling rate p(fₐ|y, m) according to the age of men. Expression 2-7 above shows the falling rate p(fₐ|y, f) according to the age of women.

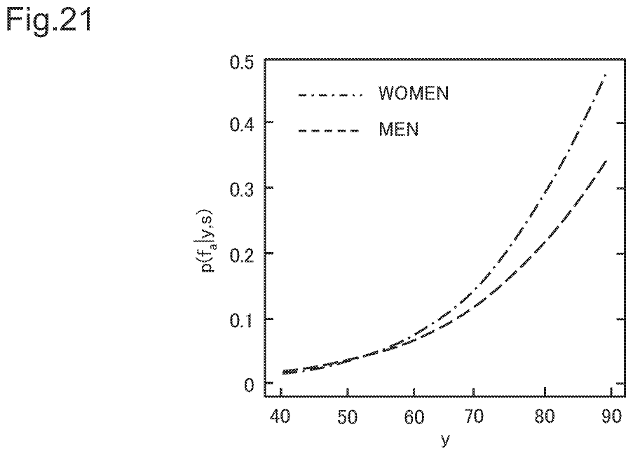

FIG. 21 is a graph relating to Expressions 2-6 and 2-7 derived by the above procedure. In FIG. 21, a curve relating to men is indicated by a broken line (Expression 2-6), and a curve relating to women is indicated by a dash-dot line (Expression 2-7). By using the relationship (Expression 2-6, Expression 2-7) in the graph of FIG. 21, the falling rate p(fₐ|y, s) according to the age y/gender s can be estimated. For example, physical data such as the age y and the gender s is input via an input device (not illustrated).

The estimation device 22 calculates the walking speed v relevant to the stride length per unit time based on the time-series data (walking waveform) of the sensor data measured by the measurement device 21. The estimation device 22 calculates a stride time relevant to the time of the stance phase in one gait cycle. The estimation device 22 calculates a mean value and a standard deviation of the walking speed v and the walking variation w for several steps. The estimation device 22 calculates the denominator p(v, w|y, s) of Expression 2-1 based on the age y and gender of the user, the calculated mean value and standard deviation of the walking speed v and the walking variation w.

The estimation device 22 applies the age y and gender s of the user, the calculated walking speed v and walking variation w, and the like to the above Expression 2-1 to estimate the falling easiness fₐ of the user. The estimation device 22 outputs information regarding the estimated falling easiness. The output destination of the information regarding the falling easiness is not particularly limited. For example, the estimation device 22 outputs information regarding the falling easiness of the user to an external system or device (not illustrated). For example, the estimation device 22 outputs information regarding the falling easiness of the user to a display device (not illustrated).

The estimation device 22 may calculate an easy falling age as the care-related information. The easy falling age is an age relevant to a measure of the falling easiness estimated using gait parameters and physical data. The estimation device 22 calculates an easy falling age Yₑ based on the following Expression 2-8.

$$p ( {f}_{a} \vert y , s , v , w ) = p ( {f}_{a} \vert {Y}_{e} ) \qquad ( 2 - 8 )$$

The left side p(fₐ|y, s, v, w) of the above Expression 2-8 is the falling easiness according to the age y, the gender s, the walking speed v, and the walking variation w. The right side p(fₐ|Yₑ) of Expression 2-8 is the falling easiness according to the easy falling age Yₑ. The right side p(fₐ|Yₑ) of Expression 2-8 is a function related to the age and stride time variation in the frequency distribution of FIG. 19.

The estimation device 22 calculates the easy falling age Yₑ that satisfies the relationship of Expression 2-8 described above. The estimation device 22 applies the value obtained by the above Expression 2-1 to the left side p(fₐ|y, s, v, σ) of Expression 2-8. The estimation device 22 calculates the easy falling age Yₑ that is the same as the value on the left side with respect to the function related to the age and the stride time variation in the frequency distribution of FIG. 19. For example, the estimation device 22 outputs the calculated information regarding the easy falling age as the care-related information.

### [Estimation Device]

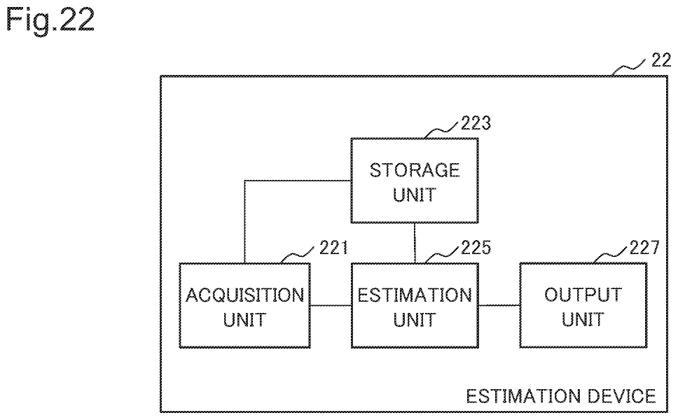

Next, details of the estimation device 22 will be described with reference to the drawings. FIG. 22 is a block diagram illustrating an example of the detailed configuration of the estimation device 22. The estimation device 22 includes an acquisition unit 221, a storage unit 223, an estimation unit 225, and an output unit 227.

The acquisition unit 221 has a configuration similar to that of the acquisition unit 121 according to the first example embodiment. The acquisition unit 221 acquires data (also referred to as physical data) such as the age and gender of the user input via an input device (not illustrated). The acquisition unit 221 stores the acquired physical data in the storage unit 223. The acquisition unit 221 receives sensor data from the measurement device 21. The acquisition unit 221 outputs the received sensor data to the estimation unit 225.

The storage unit 223 stores an estimation model for estimating the falling easiness based on the age, gender, walking speed distribution, and walking variation distribution. The estimation model is constructed based on past knowledge. The estimation model outputs the falling easiness according to the walking speed distribution and the walking variation distribution calculated using the sensor data measured as the user walks, and the inputs of the age and gender of the user. The storage unit 223 stores the physical data of the user. The physical data includes the age and gender of the subject. The physical data may include data such as the height and weight of the user.

The estimation unit 225 has a configuration similar to that of the estimation unit 125 according to the first example embodiment. The estimation unit 225 acquires the physical data regarding the user from the storage unit 223. The estimation unit 225 acquires, from the acquisition unit 221, sensor data measured by the measurement device 21 installed on the footwear worn by the user. The estimation unit 225 generates time-series data of the sensor data. The estimation unit 225 extracts walking waveform data for at least one gait cycle from the generated time-series data. The estimation unit 225 converts the coordinate system of the acquired sensor data from the local coordinate system to the world coordinate system, and generates time-series data (also referred to as a walking waveform) of the sensor data after conversion to the world coordinate system.

The estimation unit 225 detects a walking event from the generated walking waveform. The estimation unit 225 calculates the stride length of the user based on the detected walking event. The estimation unit 225 calculates the walking speed of the user by dividing the stride length by the time relevant to one stride. A method of calculating the walking speed is similar to that of the first example embodiment. The estimation unit 225 calculates a stride time relevant to the time of the stance phase in one gait cycle. The stride time corresponds to the time from the heel strike to the toe off.

The estimation unit 225 calculates a mean value and a standard deviation of the walking speed v and the walking variation w for several steps. For example, the estimation unit 225 calculates the mean value and the standard deviation of the walking speed v and the walking variation w using the sensor data for 3 to 10 steps.

The estimation unit 225 inputs the physical data (age, gender) of the user and the numerical values regarding the walking speed distribution and the walking variation distribution calculated based on the walking of the user to the estimation model for estimating the falling easiness stored in the storage unit 223. The estimation unit 225 outputs the falling easiness output from the estimation model to the output unit 227 according to the inputs of the physical data, the walking speed distribution, and the walking variation distribution.

For example, the estimation unit 225 may estimate the easy falling age of the user based on the age of the user and the walking speed distribution/walking variation distribution. The estimation unit 225 inputs the physical data (age, gender) of the user and the numerical values regarding the walking speed distribution/walking variation distribution calculated based on the walking of the user to the estimation model for estimating the easy falling age stored in the storage unit 223. The estimation unit 225 outputs the easy falling age from the estimation model to the output unit 227 as the care-related information according to the inputs of the numerical value related to the walking speed distribution/walking variation distribution and the physical data.

The output unit 227 has a configuration similar to that of the output unit 127 according to the first example embodiment. The output unit 227 outputs the estimated care-related information. The output destination (not illustrated) of the care-related information is not particularly limited. The output care-related information can be used for any purpose.

### (Operation)

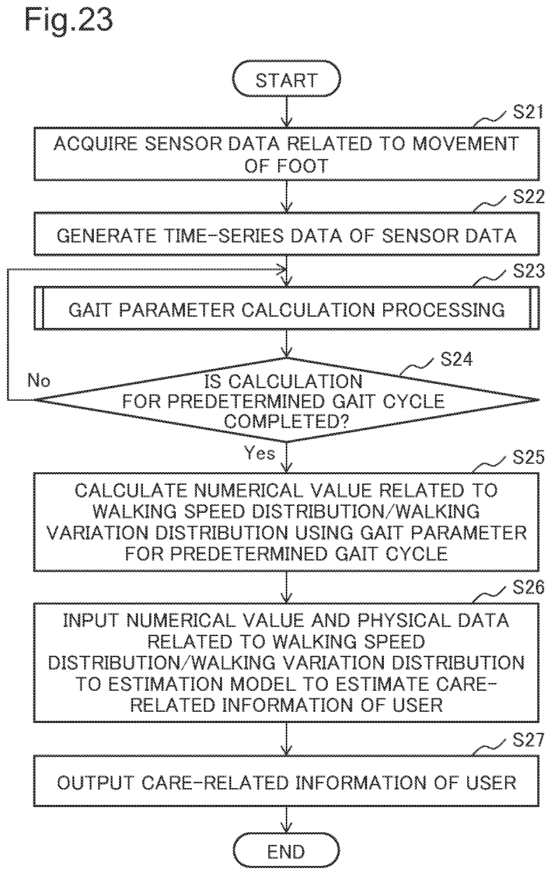

Next, an operation of the information presentation system 2 will be described with reference to the drawings. Hereinafter, the operation of the measurement device 21 will not be described, and the operation of the estimation device 22 will be described. FIG. 23 is a flowchart for explaining an example of the operation of the estimation device 22. In the description along the flowchart of FIG. 23, the estimation device 22 will be described as an operation subject.

In the flowchart of FIG. 23, first, the estimation device 22 acquires sensor data regarding the movement of the foot (step S21). The estimation device 22 acquires sensor data regarding the movement of the foot from the measurement device 21. The estimation device 22 may acquire the sensor data measured by the measurement device 21 for each gait cycle, or may collectively acquire the sensor data for a plurality of gait cycles.

Next, the estimation device 22 generates time-series data of sensor data (step S22). The estimation device 22 generates time-series data of sensor data regarding accelerations and trajectories in three axis directions, angular velocities around three axes, and the like. In the present example embodiment, the estimation device 22 generates time-series data of sensor data regarding acceleration and a trajectory in the Y direction.

Next, the estimation device 22 executes gait parameter calculation processing (step S23). In the gait parameter calculation processing, the estimation device 22 calculates a walking speed and a stride time based on time-series data (walking waveform) for one gait cycle. Details of the gait parameter calculation processing will be described later.

When the calculation for the predetermined gait cycle is completed (Yes in step S24), the estimation device 22 derives a numerical value related to the walking speed distribution/walking variation distribution by using the gait parameter for the predetermined gait cycle (step S25). The numerical value related to the walking speed distribution/walking variation distribution includes a mean value and a standard deviation of the walking speed/walking variation in walking for a predetermined gait cycle. When the calculation for the predetermined gait cycle is not completed (No in step S24), the process returns to step S23.

The estimation device 22 inputs the numerical value and the physical data regarding the walking speed distribution/walking variation distribution to the estimation model to estimate the care-related information of the user (step S26). For example, the estimation device 22 estimates the falling easiness and the easy falling age as the care-related information.

The estimation device 22 outputs the care-related information of the user (step S27). For example, the estimation device 22 outputs the falling easiness and the easy falling age as the care-related information. There is no particular limitation on the output destination and use of the care-related information of the user.

### [Gait Parameter Calculation Processing]

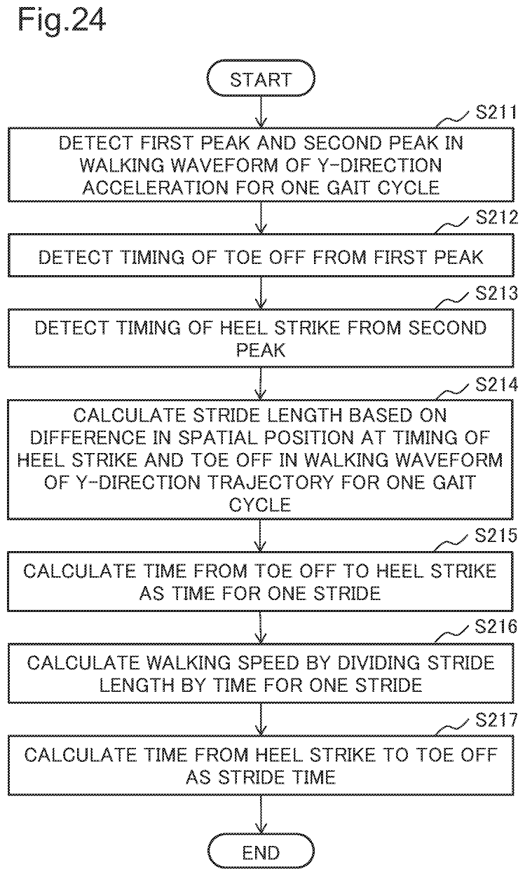

Next, the gait parameter calculation processing by the estimation device 22 will be described with reference to the drawings. FIG. 24 is a flowchart for explaining an example of the gait parameter calculation processing. In the description along the flowchart of FIG. 24, the estimation device 22 will be described as an operation subject.

In FIG. 24, first, the estimation device 22 detects the first peak and the second peak in the walking waveform of the Y-direction acceleration (traveling direction acceleration) for one gait cycle (step S211).

Next, the estimation device 22 detects the timing of the toe off from the first peak (step S212).

Next, the estimation device 22 detects the timing of heel strike from the second peak (step S213).

Next, the estimation device 22 calculates the stride length based on the difference in the spatial position at the timing of the heel strike and the toe off in the walking waveform of the Y-direction trajectory (traveling direction trajectory) for one gait cycle (step S214).

Next, the estimation device 22 calculates the time from the toe off to the heel strike as the time for one stride (step S215).

Next, the estimation device 22 calculates the walking speed by dividing the stride length by the time relevant to one stride (step S216).

Next, the estimation device 22 calculates the time from the heel strike to the toe off as the stride time (step S217). Step S217 may be performed before steps S214 to S216, or may be performed in parallel with steps S214 to S216.

### Application Example 2

Next, Application Example 2 of the information presentation system 2 of the present example embodiment will be described with reference to the drawings. In the present application example, the information regarding the falling easiness estimated by the estimation device 22 is displayed on the screen of a mobile terminal 210. In the present application example, it is assumed that the measurement device 21 is installed in the shoe 200 of the user, and the sensor data based on the physical quantity related to the movement of the foot measured by the measurement device 21 is transmitted to the mobile terminal 210 possessed by the user. It is assumed that the sensor data transmitted to the mobile terminal 210 is subjected to data processing by a program (estimation device 22) installed in the mobile terminal 210.

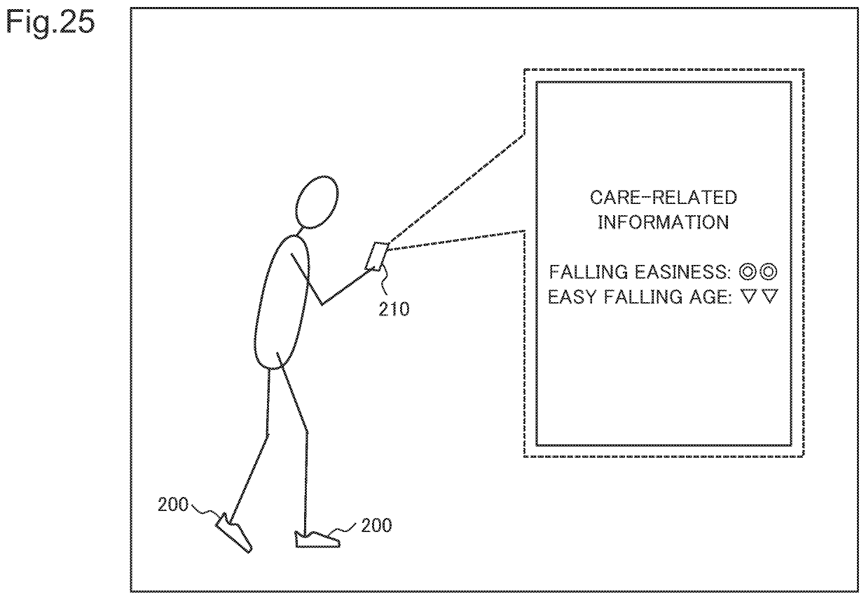

FIG. 25 is an example in which information regarding the physical condition of the user is displayed on the screen of the mobile terminal 210 of the user wearing the shoe 200 on which the measurement device 21 is installed. In the example of FIG. 25, as the care-related information, the falling easiness and the easy falling age estimated based on the walking of the user are displayed on the screen of the mobile terminal 210 of the user. The user who has browsed the care-related information displayed on the screen of the mobile terminal 210 can take an action according to the information. For example, a pedestrian who has browsed the information regarding the falling easiness displayed on the screen of the mobile terminal 210 can consult a medical institution, a nursing care facility, or the like according to the information.

As described above, the information presentation system of the present example embodiment includes the measurement device and the estimation device. The measurement device is disposed on the user's footwear. The measurement device measures the spatial acceleration and the spatial angular velocity according to the gait of the user. The measurement device generates sensor data based on the measured spatial acceleration and spatial angular velocity. The measurement device outputs the generated sensor data to the estimation device, and the estimation device includes an acquisition unit, a storage unit, an estimation unit, and an output unit. The acquisition unit acquires sensor data measured according to walking of the user and physical data of the user. The storage unit stores an estimation model that outputs the care-related information according to the inputs of the numerical value related to the distribution of the walking speed and the walking variation and the physical data. The estimation unit generates a walking waveform for a predetermined gait cycle by using time-series data of sensor data for the predetermined gait cycle of the user. The estimation unit calculates a predetermined gait parameter for each gait cycle based on the walking event detected from the walking waveform for the predetermined gait cycle. The estimation unit calculates the walking speed of the user using the calculated predetermined gait parameter. The estimation unit calculates a numerical value related to the distribution of the walking speed measured according to the walking of the user in the predetermined gait cycle. The estimation unit calculates the walking variation in the stance phase of the user using the calculated predetermined gait parameter. The estimation unit calculates a numerical value related to the distribution of the walking variation measured according to the walking of the user in the predetermined gait cycle. The estimation unit inputs the calculated numerical value related to the distribution of the walking variation, the numerical value related to the distribution of the walking speed, and the physical data to the estimation model. The estimation unit estimates the care-related information of the user. The output unit outputs the estimated care-related information of the user.

The information presentation system according to the present example embodiment uses an estimation model that outputs care-related information according to the inputs of the numerical values regarding distributions of walking speed and walking variation and the physical data. Therefore, according to the information presentation system of the present example embodiment, it is possible to estimate the care-related information according to the physical condition of the user based on the numerical value regarding the distribution of the walking speed and the walking variation and the physical data.

In one aspect of the present example embodiment, the estimation unit generates a walking waveform of the acceleration in the traveling direction and a walking waveform of the trajectory in the traveling direction by using sensor data for a predetermined gait cycle of the user. The estimation unit detects the heel strike and the toe off from the walking waveform of the acceleration in the traveling direction as a walking event. The estimation unit calculates the time from the heel strike to the toe off as the stride time. According to the present aspect, the stride time can be calculated based on the time from the heel strike to the toe off detected from the walking waveform of the acceleration in the traveling direction.

In one aspect of the present example embodiment, the acquisition unit acquires the age and gender of the user as physical data. The storage unit stores an estimation model that outputs the falling easiness as the care-related information according to the inputs of the numerical value related to the distribution of the walking speed and the walking variation and the age and attribute of the user. The estimation unit inputs numerical values related to the distribution of the walking speed and the walking variation, and the age and attribute of the user to the estimation model to estimate the falling easiness of the user. According to the present aspect, the falling easiness of the user can be estimated based on the numerical values related to the distribution of the walking speed and the walking variation of the user and the age and gender of the user.

In one aspect of the present example embodiment, the estimation unit estimates the frail age of the user based on the estimated falling easiness and the correlation between age and falling easiness. According to the present aspect, the easy falling age of the user can be estimated based on the falling easiness of the user.

### Third Example Embodiment

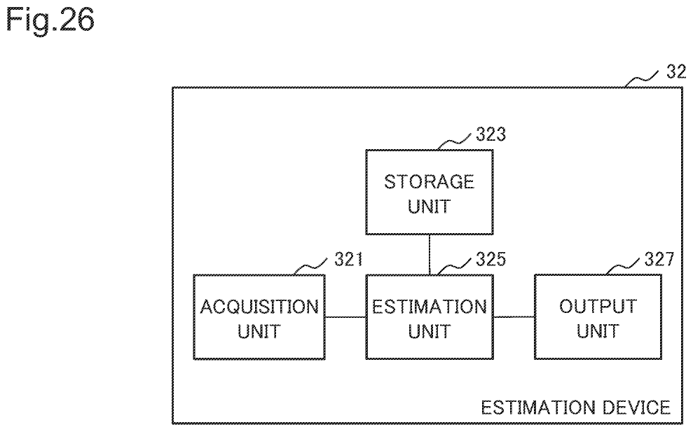

Next, an estimation device according to a third example embodiment will be described with reference to the drawings. The estimation device of the present example embodiment has a configuration in which the estimation devices included in the information presentation systems of the first and second example embodiments are simplified. FIG. 26 is a block diagram illustrating a configuration of an estimation device 32 according to the present example embodiment. The estimation device 32 includes an acquisition unit 321, a storage unit 323, an estimation unit 325, and an output unit 327.

The acquisition unit 321 acquires sensor data measured according to walking of the user and physical data of the user. The storage unit 323 stores the estimation model that outputs the care-related information according to the inputs of the feature quantity extracted from the sensor data and the physical data, and the physical data. The estimation unit 325 inputs the feature quantity and the physical data extracted from the sensor data of the user to the estimation model, and estimates the care-related information of the user. The output unit 327 outputs the estimated care-related information of the user.

As described above, the estimation device of the present example embodiment can estimate the care-related information according to the physical condition of the user based on the gait of the user by using the estimation model that outputs the care-related information according to the inputs of the feature quantity extracted from the sensor data and the physical data.

### (Hardware)

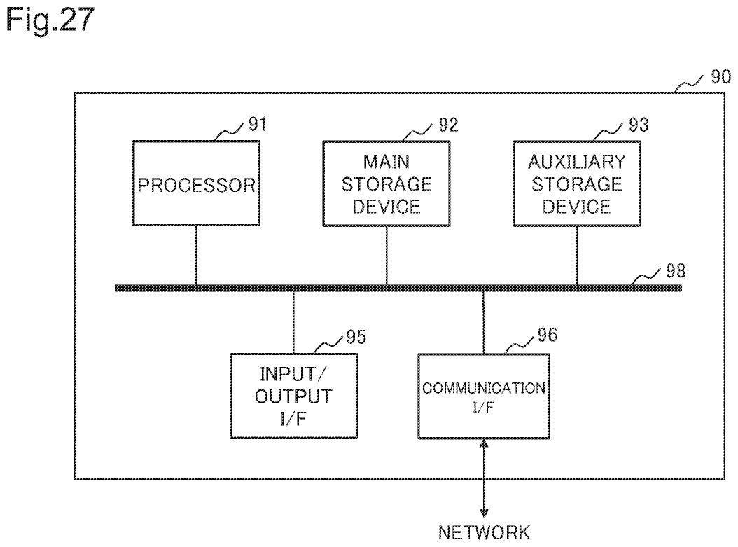

Here, a hardware configuration for executing processing of the control unit according to each example embodiment of the present disclosure will be described using an information processing device 90 of FIG. 27 as an example. The information processing device 90 in FIG. 27 is a configuration example for executing the control and processing of each example embodiment, and does not limit the scope of the present disclosure.

As illustrated in FIG. 27, the information processing device 90 includes a processor 91, a main storage device 92, an auxiliary storage device 93, an input/output interface 95, and a communication interface 96. In FIG. 27, the interface is abbreviated as an I/F. The processor 91, the main storage device 92, the auxiliary storage device 93, the input/output interface 95, and the communication interface 96 are data-communicably connected to each other via a bus 98. The processor 91, the main storage device 92, the auxiliary storage device 93, and the input/output interface 95 are connected to a network such as the Internet or an intranet via the communication interface 96.

The processor 91 develops a program stored in the auxiliary storage device 93 or the like in the main storage device 92. The processor 91 executes the program developed in the main storage device 92. In the present example embodiment, a software program installed in the information processing device 90 may be used. The processor 91 executes processing or control according to each example embodiment.

The main storage device 92 has an area in which a program is developed. A program stored in the auxiliary storage device 93 or the like is developed in the main storage device 92 by the processor 91. The main storage device 92 is implemented by, for example, a volatile memory such as a dynamic random access memory (DRAM). A nonvolatile memory such as a magnetoresistive random access memory (MRAM) may be configured and added as the main storage device 92.

The auxiliary storage device 93 stores various data such as programs. The auxiliary storage device 93 is implemented by a local disk such as a hard disk or a flash memory. Various data may be stored in the main storage device 92, and the auxiliary storage device 93 may be omitted.

The input/output interface 95 is an interface for connecting the information processing device 90 and a peripheral device. The communication interface 96 is an interface for connecting to an external system or device through a network such as the Internet or an intranet based on a standard or a specification. The input/output interface 95 and the communication interface 96 may be shared as an interface connected to an external device.

An input device such as a keyboard, a mouse, or a touch panel may be connected to the information processing device 90 as necessary. These input devices are used to input information and settings. When the touch panel is used as an input device, the display screen of the display device may also serve as the interface of the input device. Data communication between the processor 91 and the input device may be mediated by the input/output interface 95.

The information processing device 90 may be provided with a display device for displaying information. In a case where a display device is provided, the information processing device 90 may include a display control device (not illustrated) for controlling display of the display device. The display device may be connected to the information processing device 90 via the input/output interface 95.

The information processing device 90 may be provided with a drive device. The drive device mediates reading of data and a program from a recording medium, writing of a processing result of the information processing device 90 to the recording medium, and the like between the processor 91 and the recording medium (program recording medium). The drive device may be connected to the information processing device 90 via the input/output interface 95.

The above is an example of the hardware configuration for enabling the control and processing according to each example embodiment of the present invention. The hardware configuration of FIG. 27 is an example of a hardware configuration for executing the control and processing of each example embodiment, and does not limit the scope of the present invention. A program for causing a computer to execute the control and processing according to each example embodiment is also included in the scope of the present invention. Further, a program recording medium in which the program according to each example embodiment is recorded is also included in the scope of the present invention. The recording medium can be achieved by, for example, an optical recording medium such as a compact disc (CD) or a digital versatile disc (DVD). The recording medium may be implemented by a semiconductor recording medium such as a universal serial bus (USB) memory or a secure digital (SD) card. The recording medium may be implemented by a magnetic recording medium such as a flexible disk, or another recording medium. When a program executed by the processor is recorded in a recording medium, the recording medium is associated to a program recording medium.

The components of each example embodiment may be arbitrarily combined. The components of each example embodiment may be implemented by software or may be implemented by a circuit.

Although the present invention has been described with reference to the example embodiments, the present invention is not limited to the above example embodiments. Various modifications that can be understood by those skilled in the art can be made to the configuration and details of the present invention within the scope of the present invention.

Some or all of the above example embodiments may be described as the following Supplementary Notes, but are not limited to the following.

### (Supplementary Note 1)

An estimation device including:
an acquisition unit that acquires sensor data measured according to walking of a user and physical data of the user;
a storage unit that stores an estimation model that outputs care-related information according to inputs of a feature quantity extracted from the sensor data and the physical data, and the physical data;
an estimation unit that inputs a feature quantity extracted from the sensor data of the user and the physical data to the estimation model to estimate the care-related information of the user; and
an output unit that outputs the estimated care-related information of the user.

### (Supplementary Note 2)

The estimation device according to Supplementary Note 1, in which
the storage unit is configured to:
store the estimation model that outputs the care-related information according to inputs of a numerical value related to a distribution of walking speeds and the physical data, and
the estimation unit is configured to:
generate a walking waveform for a predetermined gait cycle by using time-series data of the sensor data for the predetermined gait cycle of the user;
calculate a predetermined gait parameter for each gait cycle based on a walking event detected from the walking waveform for the predetermined gait cycle;
calculate a walking speed of the user by using the calculated predetermined gait parameter;
calculate a numerical value related to a distribution of the walking speed measured according to walking of the user in the predetermined gait cycle;
input a numerical value related to the calculated distribution of the walking speed and the physical data to the estimation model; and
estimate the care-related information of the user.

### (Supplementary Note 3)

The estimation device according to Supplementary Note 2, in which
the estimation unit is configured to:
generate the walking waveform of acceleration in a traveling direction and the walking waveform of a trajectory in a traveling direction using the sensor data for the predetermined gait cycle of the user;
detect a toe off and a heel strike as the walking event from the walking waveform of the acceleration in the traveling direction;
extract a spatial position at a timing of the toe off and the heel strike in the walking waveform of the trajectory in the traveling direction;
calculate, as a stride length of the user, a difference between the extracted spatial positions at a timing of the toe off and the heel strike;
calculate a time from the toe off to the heel strike as a time of one stride; and
calculate the walking speed by dividing the time for one stride by the stride length.

### (Supplementary Note 4)

The estimation device according to Supplementary Note 2 or 3, in which
the acquisition unit is configured to:
acquire an age of the user as the physical data,
the storage unit is configured to:
store the estimation model that outputs a frailty probability as the care-related information according to inputs of a numerical value related to a distribution of the walking speed and an age of the user, and
the estimation unit is configured to:
input a numerical value related to a distribution of the walking speed and an age of the user to the estimation model to estimate the frailty probability of the user.

### (Supplementary Note 5)

The estimation device according to Supplementary Note 4, in which
the estimation unit is configured to:
estimate a frail age of the user based on the estimated frailty probability and a correlation between an age and a frailty prevalence.

### (Supplementary Note 6)

The estimation device according to Supplementary Note 1, in which
the storage unit is configured to:
store the estimation model that outputs the care-related information according to inputs of a numerical value related to a distribution of a walking speed and a walking variation and the physical data, and
the estimation unit is configured to:
generate a walking waveform for a predetermined gait cycle by using time-series data of the sensor data for the predetermined gait cycle of the user;
calculate a predetermined gait parameter for each gait cycle based on a walking event detected from the walking waveform for the predetermined gait cycle;
calculate a walking speed of the user by using the calculated predetermined gait parameter;
calculate a numerical value related to a distribution of the walking speed measured according to the walking of the user in the predetermined gait cycle;
calculate the walking variation in the stance phase of the user using the calculated predetermined gait parameter;
calculate a numerical value related to a distribution of the walking variation measured according to walking of the user in the predetermined gait cycle;
input a numerical value related to the calculated distribution of the walking variation, a numerical value related to a distribution of the walking speed, and the physical data to the estimation model; and
estimate the care-related information of the user.

### (Supplementary Note 7)

The estimation device according to Supplementary Note 6, in which
the estimation unit is configured to:
generate the walking waveform of an acceleration in a traveling direction and the walking waveform of a trajectory in a traveling direction using the sensor data for the predetermined gait cycle of the user;
detect, as the walking event, a heel strike and a toe off from the walking waveform of the acceleration in the traveling direction; and
calculate a time from the heel strike to the toe off as a stride time.

### (Supplementary Note 8)

The estimation device according to Supplementary Note 6 or 7, in which
the acquisition unit is configured to:
acquire an age and a gender of the user as the physical data,
the storage unit is configured to:
store the estimation model that outputs falling easiness as the care-related information according to inputs of the numerical value related to the distribution of the walking speed and the walking variation and the age and attribute of the user, and
the estimation unit is configured to:
input a numerical value related to a distribution of the walking speed and the walking variation and an age and an attribute of the user to the estimation model to estimate the falling easiness of the user.

### (Supplementary Note 9)

The estimation device according to Supplementary Note 8, in which
the estimation unit is configured to:
estimate a frail age of the user based on the estimated falling easiness and a correlation between an age and the falling easiness.

### (Supplementary Note 10)

The estimation device according to any one of Supplementary Notes 1 to 9, in which
the estimation unit is configured to:
output the care-related information to a terminal device having a screen; and
display the care-related information on the screen of the terminal device.

### (Supplementary Note 11)

An information presentation system including:
an estimation device according to any one of Supplementary Notes 1 to 10; and
a measurement device that is disposed on footwear of a user, measures a spatial acceleration and a spatial angular velocity according to walking of the user, generates sensor data based on the measured spatial acceleration and spatial angular velocity, and outputs the generated sensor data to the estimation device.

### (Supplementary Note 12)

An estimation method causing a computer to execute:
acquiring sensor data measured according to walking of a user and physical data of the user;
inputting a feature quantity extracted from the sensor data of the user and the physical data to an estimation model that outputs care-related information according to inputs of the feature quantity extracted from the sensor data and the physical data to estimate the care-related information of the user; and
outputting the estimated care-related information of the user.

### (Supplementary Note 13)

A program causing a computer to execute:
acquiring sensor data measured according to walking of a user and physical data of the user;
inputting a feature quantity extracted from the sensor data of the user and the physical data to an estimation model that outputs care-related information according to inputs of the feature quantity extracted from the sensor data and the physical data to estimate the care-related information of the user; and
outputting the estimated care-related information of the user.

### REFERENCE SIGNS LIST

1, 2 information presentation system
11, 21 measurement device
12, 22, 32 estimation device
100, 200 shoe
110, 210 mobile terminal
111 acceleration sensor
112 angular velocity sensor
113 control unit
115 transmission unit
121, 221, 321 acquisition unit
123, 223, 323 storage unit
125, 225, 325 estimation unit
127, 227, 327 output unit

What is claimed is:

1. An estimation device comprising:
a memory storing instructions; and
a processor connected to the memory and configured to execute the instructions to:
acquire sensor data measured according to walking of a user and physical data of the user;
input a feature quantity extracted from the sensor data of the user and the physical data to an estimation model that outputs care-related information in response to inputs of the feature quantity extracted from the sensor data and the physical data to estimate the care-related information of the user; and
output the estimated care-related information of the user.

2. The estimation device according to claim 1, wherein
the estimation model is configured to output the care-related information in response to inputs of a numerical value related to a distribution of walking speeds and the physical data, and
the processor is configured to execute the instructions to
generate a walking waveform for a predetermined gait cycle by using time-series data of the sensor data for the predetermined gait cycle of the user,
calculate a predetermined gait parameter for each gait cycle based on a walking event detected from the walking waveform for the predetermined gait cycle,
calculate a walking speed of the user by using the calculated predetermined gait parameter,
calculate a numerical value related to a distribution of the walking speed measured according to walking of the user in the predetermined gait cycle,
input a numerical value related to the calculated distribution of the walking speed and the physical data to the estimation model, and
estimate the care-related information of the user.

3. The estimation device according to claim 2, wherein
the processor is configured to execute the instructions to
generate the walking waveform of acceleration in a traveling direction and the walking waveform of a trajectory in a traveling direction using the sensor data for the predetermined gait cycle of the user,
detect a toe off and a heel strike as the walking event from the walking waveform of the acceleration in the traveling direction,
extract a spatial position at a timing of the toe off and the heel strike in the walking waveform of the trajectory in the traveling direction,
calculate, as a stride length of the user, a difference between the extracted spatial positions at a timing of the toe off and the heel strike,
calculate a time from the toe off to the heel strike as a time of one stride, and
calculate the walking speed by dividing the time for one stride by the stride length.

4. The estimation device according to claim 2, wherein
the estimation model is configured to output a frailty probability as the care-related information in response to inputs of a numerical value related to a distribution of the walking speed and an age of the user, and
the processor is configured to execute the instructions to
acquire an age of the user as the physical data, and
input a numerical value related to a distribution of the walking speed and an age of the user to the estimation model to estimate the frailty probability of the user.

5. The estimation device according to claim 4, wherein
the processor is configured to execute the instructions to
estimate a frail age of the user based on the estimated frailty probability and a correlation between an age and a frailty prevalence.

6. The estimation device according to claim 1, wherein
the estimation model is configured to output the care-related information in response to inputs of a numerical value related to a distribution of a walking speed and a walking variation and the physical data, and
the processor is configured to execute the instructions to
generate a walking waveform for a predetermined gait cycle by using time-series data of the sensor data for the predetermined gait cycle of the user,
calculate a predetermined gait parameter for each gait cycle based on a walking event detected from the walking waveform for the predetermined gait cycle,
calculate a walking speed of the user by using the calculated predetermined gait parameter,
calculate a numerical value related to a distribution of the walking speed measured according to the walking of the user in the predetermined gait cycle,
calculate the walking variation in the stance phase of the user using the calculated predetermined gait parameter,
calculate a numerical value related to a distribution of the walking variation measured according to walking of the user in the predetermined gait cycle,
input a numerical value related to the calculated distribution of the walking variation, a numerical value related to a distribution of the walking speed, and the physical data to the estimation model, and
estimate the care-related information of the user.

7. The estimation device according to claim 6, wherein
the processor is configured to execute the instructions to
generate the walking waveform of an acceleration in a traveling direction and the walking waveform of a trajectory in a traveling direction using the sensor data for the predetermined gait cycle of the user,
detect, as the walking event, a heel strike and a toe off from the walking waveform of the acceleration in the traveling direction, and
calculate a time from the heel strike to the toe off as a stride time.

8. The estimation device according to claim 6, wherein
the acquisition means is configured to
acquire an age and a gender of the user as the physical data,
the estimation model is configured to output falling easiness as the care-related information in response to inputs of the numerical value related to the distribution of the walking speed and the walking variation and the age and attribute of the user, and
the processor is configured to execute the instructions to
input a numerical value related to a distribution of the walking speed and the walking variation and the age and attribute of the user to the estimation model to estimate the falling easiness of the user.

9. The estimation device according to claim 8, wherein
the processor is configured to execute the instructions to
estimate a frail age of the user based on the estimated falling easiness and a correlation between an age and the falling easiness.

10. The estimation device according to claim 1, wherein
the processor is configured to execute the instructions to
output the care-related information to a terminal device having a screen, and
display the care-related information on the screen of the terminal device.

11. An information presentation system comprising:
an estimation device according to claim 1; and
a measurement device that is disposed on footwear of a user, measures a spatial acceleration and a spatial angular velocity according to walking of the user, generates sensor data based on the measured spatial acceleration and spatial angular velocity, and outputs the generated sensor data to the estimation device.

12. An estimation method causing a computer to execute:
acquiring sensor data measured according to walking of a user and physical data of the user;
inputting a feature quantity extracted from the sensor data of the user and the physical data to an estimation model that outputs care-related information in response to inputs of the feature quantity extracted from the sensor data and the physical data to estimate the care-related information of the user; and
outputting the estimated care-related information of the user.

13. A non-transitory recording medium having stored therein a program to cause a computer to execute:
acquiring sensor data measured according to walking of a user and physical data of the user;
inputting a feature quantity extracted from the sensor data of the user and the physical data to an estimation model that outputs care-related information in response to inputs of the feature quantity extracted from the sensor data and the physical data to estimate the care-related information of the user; and
outputting the estimated care-related information of the user.

14. The estimation device according to claim 1, wherein
the estimation model is constructed by machine learning, and [0051]
the care-related information is information that supports the user in making decision about taking an action related to walking. [FIG. 25, 0155]
